(19) United States
(12) Patent Application Publication
(10) Pub. No.: US 2004/0180941 A1
(43) Pub. Date: Sep. 16, 2004

(54) 3-(1-[3-(1,3-Benzothiazol-6-yl)propylcarbamoyl]cycloalkyl)propanoic acid derivatives as NEP inhibitors

(75) Inventor: David Hepworth, Sandwich (GB)

Correspondence Address: PFIZER INC., PATENT DEPARTMENT, MS8260-1611, EASTERN POINT ROAD, GROTON, CT, 06340, US

(73) Assignee: Pfizer Inc

(21) Appl. No.: 10/800,065

(22) Filed: Mar. 12, 2004

Related U.S. Application Data
(60) Provisional application No. 60/464,608, filed on Apr. 22, 2003.
(60) Provisional application No. 60/538,079, filed on Jan. 20, 2004.

(30) Foreign Application Priority Data
Mar. 14, 2003 (GB) 0305916.9
Dec. 16, 2003 (GB) 0329143.2

(51) Int. Cl. C07D 277/62; A61K 31/428

(52) U.S. Cl. 514/367; 548/180

(57) ABSTRACT

The invention relates to inhibitors of neutral endopeptidase enzyme (NEP), uses thereof, processes for the preparation thereof, intermediates used in the preparation thereof and compositions containing said inhibitors. These inhibitors have utility in a variety of therapeutic areas including the treatment of male and female sexual dysfunction, particularly female sexual dysfunction (FSD), especially wherein the FSD is female sexual arousal disorder (FSAD).

### CROSS-REFERENCE TO RELATED APPLICATION

This application claims priority from UK Application Number 0305916.9, filed on Mar. 14, 2003, UK Application Number 0329143.2, filed on Dec. 16, 2003 and the benefit from U.S. Provisional Application No. 60/464,608, filed on Apr. 22, 2003 and U.S. Provisional Application No. 60/538,079, filed on Jan. 20, 2004.

### FIELD OF INVENTION

The invention relates to inhibitors of neutral endopeptidase enzyme (NEP), uses thereof, processes for the preparation thereof, intermediates used in the preparation thereof and compositions containing said inhibitors. These inhibitors have utility in a variety of therapeutic areas including the treatment of male and female sexual dysfunction, particularly female sexual dysfunction (FSD), especially wherein the FSD is female sexual arousal disorder (FSAD).

NEP inhibitors are disclosed in WO 91/07386, WO 91/10644, WO 02/02513, WO 02/079143 and EP 1,258,474.

The use of NEP inhibitors for treating FSD is disclosed in EP 1,097,719-A1. The use of NEP inhibitors for treating male sexual dysfunction (MSD) is disclosed in WO 02/03995.

The present invention provides a class of potent NEP inhibitors, which have the advantage of being selective for NEP over soluble secreted endopeptidase (SEP). The compounds of the present invention are also selective for NEP over ACE. In addition to their selectivity, the compounds of the present invention also possess unexpectedly good pharmacokinetic properties, in particular good oral bioavailability and suitable duration of action for in vivo efficacy.

According to a first aspect the invention provides a compound of general formula (I) or pharmaceutically acceptable salts, solvates or polymorphs thereof

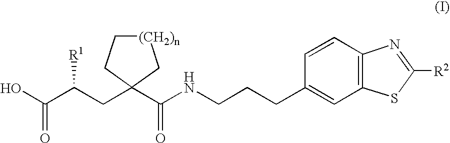

wherein

R¹ is H or CH_{3;}

R² is C₁-C₂ alkyl; and

n is 1 or 2.

A preferred aspect of the invention are compounds of formula (I) wherein n is 1.

In another preferred embodiment R¹ is methyl.

In yet another preferred embodiment R² is methyl.

A particularly preferred embodiment of the present invention are compounds of formula (I) wherein R¹ is methyl, R² is methyl and n is 1; R¹ is hydrogen, R² is ethyl and n is 1; R¹ is methyl, R² is ethyl and n is 1; and R¹ is hydrogen, R² is ethyl and n is 2.

The compounds of the invention are

(R)-2-Methyl-3-(1-{[3-(2-methyl-1,3-benzothiazol-6-yl)propyl]carbamoyl}cyclopentyl)propanoic acid (Example 1),

3-(1-{[3-(2-ethyl-1,3-benzothiazol-6-yl)propyl]carbamoyl}cyclopentyl)propanoic acid (Example 2),

(R)-2-Methyl-3-(1-{[3-(2-ethyl-1,3-benzothiazol-6-yl)propyl]carbamoyl}cyclopentyl)propanoic acid (Example 4),

3-(1-{[3-(2-ethyl-1,3-benzothiazol-6-yl)propyl]carbamoyl}cyclohexyl)propanoic acid (Example 3),

3-(1-{[3-(2-methyl-1,3-benzothiazol-6-yl)propyl]carbamoyl}cyclohexyl)propanoic acid (Example 5),

3-(1-{[3-(2-methyl-1,3-benzothiazol-6-yl)propyl]carbamoyl}cyclopentyl)propanoic acid (Example 6),

(R)-2-Methyl-3-(1-{[3-(2-methyl-1,3-benzothiazol-6-yl)propyl]carbamoyl}cyclohexyl)propanoic acid, and

(R)-2-Methyl-3-(1-{[3-(2-ethyl-1,3-benzothiazol-6-yl)propyl]carbamoyl}cyclohexyl)propanoic acid.

Preferred compounds of the invention are

(R)-2-Methyl-3-(1-{[3-(2-methyl-1,3-benzothiazol-6-yl)propyl]carbamoyl}cyclopentyl)propanoic acid (Example 1),

3-(1-{[3-(2-ethyl-1,3-benzothiazol-6-yl)propyl]carbamoyl}cyclopentyl)propanoic acid (Example 2),

(R)-2-Methyl-3-(1-{[3-(2-ethyl-1,3-benzothiazol-6-yl)propyl]carbamoyl}cyclopentyl)propanoic acid (Example 4),

3-(1-{[3-(2-ethyl-1,3-benzothiazol-6-yl)propyl]carbamoyl}cyclohexyl)propanoic acid (Example 3),

3-(1-{[3-(2-methyl-1,3-benzothiazol-6-yl)propyl]carbamoyl}cyclohexyl)propanoic acid (Example 5), and

3-(1-{[3-(2-methyl-1,3-benzothiazol-6-yl)propyl]carbamoyl}cyclopentyl)propanoic acid (Example 6).

Most preferred compounds are:

(R)-2-Methyl-3-(1-{[3-(2-methyl-1,3-benzothiazol-6-yl)propyl]carbamoyl}cyclopentyl)propanoic acid (Example 1),

3-(1-{[3-(2-ethyl-1,3-benzothiazol-6-yl)propyl]carbamoyl}cyclopentyl)propanoic acid (Example 2),

(R)-2-Methyl-3-(1-{[3-(2-ethyl-1,3-benzothiazol-6-yl)propyl]carbamoyl}cyclopentyl)propanoic acid (Example 4), and

3-(1-{[3-(2-ethyl-1,3-benzothiazol-6-yl)propyl]carbamoyl}cyclohexyl)propanoic acid (Example 3).

Pharmaceutically acceptable salts of the compounds of formula (I) include the acid addition and base salts (including disalts) thereof.

Suitable acid addition salts are formed from acids which form non-toxic salts. Examples include the acetate, aspartate, benzoate, besylate, bicarbonate/carbonate, bisulphate, camsylate, citrate, edisylate, esylate, fumarate, gluceptate, gluconate, glucuronate, hibenzate, hydrochloride/chloride, hydrobromide/bromide, hydroiodide/iodide, hydrogen phosphate, isethionate, D- and L-lactate, malate, maleate, malonate, mesylate, methylsulphate, 2-napsylate, nicotinate, nitrate, orotate, palmoate, phosphate, saccharate, stearate, succinate sulphate, D- and L-tartrate, and tosylate salts.

Suitable base salts are formed from bases which form non-toxic salts. Examples include the aluminium, ammonium, arginine, benzathine, calcium, choline, diethylamine, diolamine, glycine, lysine, magnesium, meglumine, olamine, potassium, sodium, tromethamine and zinc salts.

For a review on suitable salts, see Stahl and Wermuth, Handbook of Pharmaceutical Salts: Properties, Selection, and Use, Wiley-VCH, Weinheim, Germany (2002).

A pharmaceutically acceptable salt of a compound of formula (I) may be readily prepared by mixing together solutions of the compound of formula (I) and the desired acid or base, as appropriate. The salt may precipitate from solution and be collected by filtration or may be recovered by evaporation of the solvent.

Pharmaceutically acceptable solvates in accordance with the invention include hydrates and solvates wherein the solvent of crystallization may be isotopically substituted, e.g. D₂O, acetone-d₆, DMSO-d₆.

Also within the scope of the invention are clathrates, drug-host inclusion complexes wherein, in contrast to the aforementioned solvates, the drug and host are present in non-stoichiometric amounts. For a review of such complexes, see J Pharm Sci, 64 (8), 1269-1288 by Haleblian (August 1975).

Hereinafter all references to compounds of formula (I) include references to salts thereof and to solvates and clathrates of compounds of formula (I) and salts thereof.

The invention includes all polymorphs of the compounds of formula (I) as hereinbefore defined.

Also within the scope of the invention are so-called “prodrugs” of the compounds of formula (I). Thus certain derivatives of compounds of formula (I) which have little or no pharmacological activity themselves can, when metabolised upon administration into or onto the body, give rise to compounds of formula (I) having the desired activity. Such derivatives are referred to as “prodrugs”.

Prodrugs in accordance with the invention can, for example, be produced by replacing appropriate functionalities present in the compounds of formula (I) with certain moieties known to those skilled in the art as “pro-moieties” as described, for example, in “Design of Prodrugs” by H Bundgaard (Elsevier, 1985).

Finally, certain compounds of formula (I) may themselves act as prodrugs of other compounds of formula (I).

Compounds of formula (I) containing one or more asymmetric carbon atoms can exist as two or more optical isomers. Where a compound of formula (I) contains an alkenyl or alkenylene group, geometric cis/trans (or Z/E) isomers are possible, and where the compound contains, for example, a keto or oxime group, tautomeric isomerism (‘tautomerism’) may occur. It follows that a single compound may exhibit more than one type of isomerism.

Included within the scope of the present invention are all optical isomers, geometric isomers and tautomeric forms of the compounds of formula (I), including compounds exhibiting more than one type of isomerism, and mixtures of one or more thereof.

Cis/trans isomers may be separated by conventional techniques well known to those skilled in the art, for example, fractional crystallisation and chromatography.

Conventional techniques for the preparation/isolation of individual stereoisomers include the conversion of a suitable optically pure precursor, resolution of the racemate (or the racemate of a salt or derivative) using, for example, chiral HPLC, or fractional crystallisation of diastereoisomeric salts formed by reaction of the racemate with a suitable optically active acid or base, for example, tartaric acid.

The present invention also includes all pharmaceutically acceptable isotopic variations of a compound of formula (I). An isotopic variation is defined as one in which at least one atom is replaced by an atom having the same atomic number, but an atomic mass different from the atomic mass usually found in nature.

Examples of isotopes suitable for inclusion in the compounds of the invention include isotopes of hydrogen, such as ²H and ³H, carbon, such as ¹³C and ¹⁴C, nitrogen, such as ¹⁵N, oxygen, such as ¹⁷O and ¹⁸O, phosphorus, such as ³²P, sulphur, such as ³⁵S, fluorine, such as ¹⁸F, and chlorine, such as ³⁶Cl.

Substitution of the compounds of the invention with isotopes such as deuterium, i.e. ²H, may afford certain therapeutic advantages resulting from greater metabolic stability, for example, increased in vivo half-life or reduced dosage requirements, and hence may be preferred in some circumstances.

Certain isotopic variations of the compounds of formula (I), for example, those incorporating a radioactive isotope, are useful in drug and/or substrate tissue distribution studies. The radioactive isotopes tritium, i.e. ³H, and carbon-14, i.e. ¹⁴C, are particularly useful for this purpose in view of their ease of incorporation and ready means of detection.

Isotopic variations of the compounds of formula (I) can generally be prepared by conventional techniques known to those skilled in the art or by processes analogous to those described in the accompanying Examples and Preparations using appropriate isotopic variations of suitable reagents.

The compounds of formula (I) may be freeze-dried, spray-dried, or evaporatively dried to provide a solid plug, powder, or film of crystalline or amorphous material. Microwave or radio frequency drying may be used for this purpose.

Compounds of formula (I) may be prepared by the following process as described in scheme (I) below:

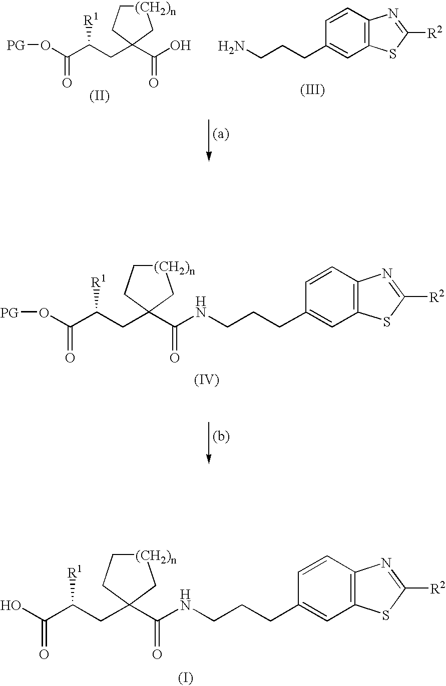

Compounds of formula (IV) may be prepared by reacting compounds of formula (II) and (III) under the conditions of process step (a) Amide bond formation—such reactions may be carried out under a wide variety of conditions well known to the skilled man.

Typically, the carboxylic acid may be activated by treatment with an agent such as 1,1′-carbonyldiimidazole (CDI), fluoro-N,N,N′,N′-tetramethylformamidinium hexafluorophosphate (TFFH), or a combination of reagents such as azabenzotriazol-1-yloxytris(pyrrolidino)phosphonium hexafluorophosphate (PyAOP) and 1-hydroxy-7-azabenzotriazole (HOAt). Alternatively, the reaction may be carried out by addition of a peptide coupling agent such as O-(7-azabenzotriazol-1-yl)-N,N,N′,N′-uranium hexafluorophosphate (HATU), or O-benzotriazol-1-yl-N,N,N′,N′-uranium hexafluorophosphate (HBTU), or N,N′-dicyclohexylcarbodiimide (DCC), 1,3-diisopropylcarbodiimide (DIC) to a mixture of the acid and amine. The reaction is carried out in a suitable solvent such as CH₂Cl₂, Pyridine, N,N-dimethylformamide (DMF), N,N-dimethylacetamide (DMA) or 1-methyl-2-pyrrolidinone between 0° C. and the boiling point of the solvent.

Preferably, the conversion is effected using CDI, triethylamine and isopropyl acetate as solvent.

The product of process step (a) is then treated under the conditions of process step (b) Removal of protecting group PG. Suitable groups are described in “Protective Groups in Organic Synthesis” by T. W. Greene and P. G. M. Wuts, John Wiley and Sons Inc, 1991.

The conditions required for removal of the protecting group are often specific to that protecting group; conditions for their removal may be found in references such as Greene T. W., Wuts, P. G. M. Protective Groups in Organic Synthesis, Wiley-Interscience and Kocienski, P. J. Protecting Groups, Thieme.

Preferably, PG is a tert-butyl group and deprotection is acid catalysed using a suitable solvent at room temperature. Preferred conditions are trifluoroacetic acid in dichloromethane.

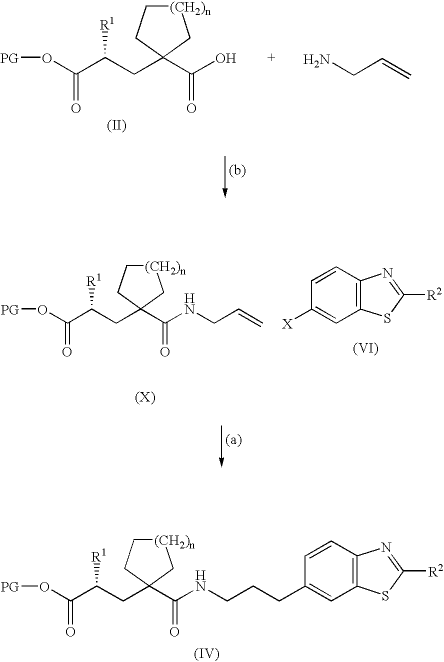

In an alternative process, compounds of formula (IV) can be prepared from compounds of formula (X) and (VI) by process steep (a) comprising an aryl-allyl coupling. Suitable conditions are well known to the person skilled in the art. Compounds of formula (X) may be prepared from compounds of formula (II) and allylamine by process step (b) comprising amide bond formation. Such reactions may be carried out under a wide variety of conditions well known to the skilled person.

Compounds of formula (II) and (III) may be prepared according to the processes described in WO02/079143 .

In addition, compounds of formula (III) may be prepared as described below:

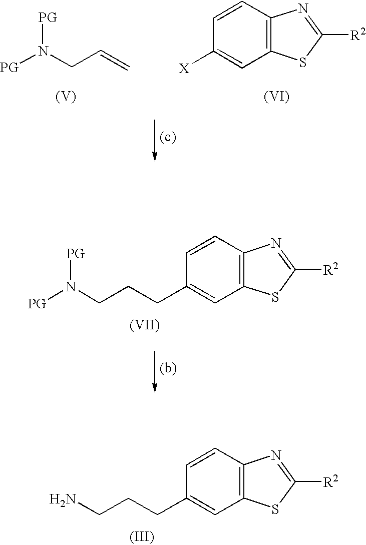

Compounds of formula (VII) may be prepared from compounds of formula (V) and (VI) under the conditions of process step (c) an aryl-allyl coupling. Suitable conditions are well known to a man skilled in the art. Particularly suitable conditions are those of the Suzuki-Miyaura coupling reaction [Angew. Chem. Int. Ed. 2001, 40(24), 4544-4568] with a hydroborated intermediate, prepared from an appropriately protected allylamine derivative (e.g. Di(tert-butyl) allylimidodicarbonate Bioorganic & Medicinal Chemistry Letters, 1999, 7, 1625-1636) and a borane derivative, such as 9-BBN.

Compounds of formula (III) may be prepared from compounds of formula (VII) under the conditions of process step (b) Removal of protecting group PG described herein.

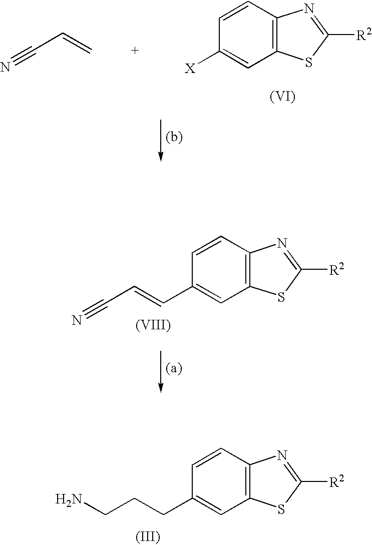

Alternatively, amine (III) can be prepared from compounds of formula (VIII) by process step (a) comprising reduction of carbon-carbon and carbon-nitrogen multiple bonds. Suitable conditions are well known to the person skilled in the art. Particularly suitable conditions comprise treatment with Boc₂O, NiCl₂ and NaBH₄ followed by deprotection of the resultant tertiary butyl group as described above.

Compounds of formula (VIII) can be prepared from acrylonitrile and compounds of formula (VI) by process step (b) comprising an aryl-allyl coupling where X is a halogen, preferably iodine. Suitable conditions are well known to the person skilled in the art. Particularly suitable conditions comprise treatment with Pd(OAc)₂, P(o-tolyl)₃, and NaOAc in DMF.

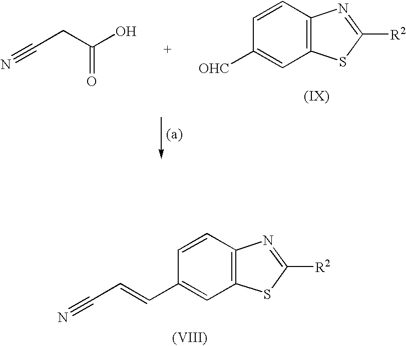

Alternatively, compounds of formula (VIII) may be prepared from cyanoacetic acid and compounds of formula (IX) by process step (a) condensation. Suitable conditions are well known to a man skilled in the art. Suitable conditions are described in “Advanced Organic Chemistry” by Jerry March, 4^{th} edition, Wiley-Interscience, p. 945. Compounds of formula (IX) are known in the literature (e.g. Zhurnal Obshchei Khimii (1964), 34(11), 3801-6).

All of the above reactions and the preparations of novel starting materials used in the preceding methods are conventional. Appropriate reagents and reaction conditions for their performance or preparation as well as procedures for isolating the desired products will be well-known to those skilled in the art with reference to literature precedents and the Examples and Preparations hereinbelow.

The compounds of the present invention are a class of NEP inhibitors selective for NEP over SEP.

In general it is important for a drug to be as selective as possible for its desired target enzyme; additional activities give rise to the possibility of side effects. SEP was relatively recently identified and its exact physiological role has yet to be fully determined. However, irrespective of whatever role SEP may or may not play, it is a sound medicinal chemistry precept to ensure that any drug has selectivity over closely related mechanistic targets of unknown physiological function.

Without being bound by any theory it is becoming clear that NEP and SEP are capable of hydrolysing many of the same biologically important peptides such as enkaphalin, endothelin (ET), big-endothelin (Big ET), bradykinin, substance P, angiotensin1, atrial natriuretic peptide (ANP), and gonadotropic releasing hormone (GnRH).

If a patient is treated with a drug that inhibits NEP and SEP, the hydrolysis of many of these peptides (most of which are not involved with the improvement in sexual function associated with NEP inhibition) may be reduced and the levels of these peptides will therefore increased. A number of side effects associated with a rise in the levels of these peptides may be posited: blood pressure may be lowered when ANP levels are increased; increases in enkephalin levels may lead to changes in pain perception; endothelin-1 is a potent vasoconstrictor, reducing the levels of conversion of Big ET to ET, or the hydrolysis of ET-1 may lead to changes in blood pressure.

Thus if the patient is given a NEP selective inhibitor, the increases in levels of these substrate peptides will be less since active SEP enzyme will still be present. Any side effects associated with changes in levels of these peptides will therefore also be less.

In addition the mRNA for SEP can be found in other tissues at varying levels, including in the testes, heart, brain, kidney, salivary glands, thyroid glands, placenta, small intestine and ovary (in house data and Bonvouloir et al). In the case of mice, SEP RNA has also been detected in the spleen and adrenal glands. A NEP inhibitor that does not inhibit SEP is therefore likely to have the advantage of a greater potential for a cleaner physiological profile.

Surprisingly, in identifying a class of NEP inhibitors selective over SEP, it has been discovered that these compounds have favourable pharmacokinetic properties for oral administration.

An orally administered drug should have good bioavailablity—that is an ability to readily cross the gastrointestinal (GI) tract and have a metabolic rate such that it is not subject to extensive metabolism as it passes from the GI tract into the systemic circulation. Molecules which are quickly metabolised will have lower bioavailablity as more compound will be removed by metabolism as it passes into the systemic circulation. Once a drug is in the systemic circulation the metabolic rate is also important in determining the time of residence of the drug in the body—fast metabolism of a drug will lead to it having a short duration of action.

Thus, it is clearly favourable for drug molecules to have the properties of being readily able to cross the GI tract, and being only slowly metabolised in the body.

The CACO-2 assay is a widely accepted model for predicting the ability of a given molecule to cross the GI tract. The molecules of the present invention have good CACO-2 flux.

The majority of metabolism of drug molecules generally occurs in the liver. Therefore the use of human liver microsomes (HLM) is a widely accepted method for measuring the susceptibility of a given molecule towards metabolism in the liver. The compounds of the present invention are stable towards HLM.

Compounds which have good CACO-2 flux, and are stable towards HLM are predicted to have good oral bioavailability (good absorption across the GI tract and minimal extraction of compound as it passes through the liver) and a long residence time in the body—sufficient for the drug to be efficacious.

Additionally the compounds of the invention are crystalline in the free acid form, without recourse to salt formation and are thus particularly easy to handle.

The compounds of the invention are inhibitors of the zinc-dependent, neutral endopeptidase EC.3.4.24.11., and it is proposed that the compounds of the invention will treat the disease states listed below. This enzyme is involved in the breakdown of several bioactive oligopeptides, cleaving peptide bonds on the amino side of hydrophobic amino acid residues. The peptides metabolised include atrial natriuretic peptides (ANP), bombesin, bradykinin, calcitonin gene-related peptide, endothelins, enkephalins, neurotensin, substance P and vasoactive intestinal peptide. Some of these peptides have potent vasodilatory and neurohormone functions, diuretic and natriuretic activity or mediate behaviour effects.

Thus, the compounds of the invention, by inhibiting the neutral endopeptidase EC.3.4.24.11, can potentiate the biological effects of bioactive peptides. Thus, in particular the compounds have utility in the treatment of a number of disorders, including hypertension, pulmonary hypertension, peripheral vascular disease, heart failure, angina, renal insufficiency, acute renal failure, cyclical oedema, Menieres disease, hyperaldosteroneism (primary and secondary) and hypercalciuria. The term hypertension includes all diseases characterised by supranormal blood pressure, such as essential hypertension, pulmonary hypertension, secondary hypertension, isolated systolic hypertension, hypertension associated with diabetes, hypertension associated with atherosclerosis, and renovascular hypertension, and further extends to conditions for which elevated blood pressure is a known risk factor. Accordingly, the term “treatment of hypertension” includes the treatment or prevention of complications arising from hypertension, and other associated co-morbidities, including congestive heart failure, angina, stroke, glaucoma, impaired renal function, including renal failure, obesity, and metabolic diseases (including Metabolic Syndrome). Metabolic diseases include in particular diabetes and impaired glucose tolerance, including complications thereof, such as diabetic retinopathy and diabetic neuropathy.

In addition, because of their ability to potentiate the effects of ANF the compounds have utility in the treatment of glaucoma. As a further result of their ability to inhibit the neutral endopeptidase E.C.3.4.24.11 the compounds of the invention may have activity in other therapeutic areas including for example the treatment of menstrual disorders, preterm labour, pre-eclampsia, endometriosis, and reproductive disorders (especially male and female infertility, polycystic ovarian syndrome, implantation failure). Also the compounds of the invention should treat asthma, inflammation, leukemia, pain, cancer pain, depression, drug abuse, cirrhosis, epilepsy, affective disorders, dementia and geriatric confusion, obesity and gastrointestinal disorders (especially diarrhoea and irritable bowel syndrome), wound healing (especially diabetic and venous ulcers and pressure sores), septic shock, the modulation of gastric acid secretion, the treatment of hyperreninaemia, cystic fibrosis, restenosis, diabetic complications and athereosclerosis.

In a preferred embodiment the compounds of the invention are useful in the treatment of male and female sexual dysfunction. The compounds of the invention are particularly beneficial for the treatment of FSD (especially FSAD) and male sexual dysfunction (especially male erectile dysfunction (MED)).

In accordance with the invention, FSD can be defined as the difficulty or inability of a woman to find satisfaction in sexual expression. FSD is a collective term for several diverse female sexual disorders (Leiblum, S. R. (1998). Definition and classification of female sexual disorders. Int. J. Impotence Res., 10, S104-S106; Berman, J. R., Berman, L. & Goldstein, I. (1999). Female sexual dysfunction: Incidence, pathophysiology, evaluations and treatment options. Urology, 54, 385-391). The woman may have lack of desire, difficulty with arousal or orgasm, pain with intercourse or a combination of these problems. Several types of disease, medications, injuries or psychological problems can cause FSD. Treatments in development are targeted to treat specific subtypes of FSD, predominantly desire and arousal disorders.

The categories of FSD are best defined by contrasting them to the phases of normal female sexual response: desire, arousal and orgasm (Leiblum, S. R. (1998). Definition and classification of female sexual disorders. Int. J. Impotence Res., 10, S104-S106). Desire or libido is the drive for sexual expression. Its manifestations often include sexual thoughts either when in the company of an interested partner or when exposed to other erotic stimuli. Arousal is the vascular response to sexual stimulation, an important component of which is genital engorgement and includes increased vaginal lubrication, elongation of the vagina and increased genital sensation/sensitivity. Orgasm is the release of sexual tension that has culminated during arousal.

Hence, FSD occurs when a woman has an inadequate or unsatisfactory response in any of these phases, usually desire, arousal or orgasm. FSD categories include hypoactive sexual desire disorder, sexual arousal disorder, orgasmic disorders and sexual pain disorders. Although the compounds of the invention will improve the genital response to sexual stimulation (as in female sexual arousal disorder), in doing so it may also improve the associated pain, distress and discomfort associated with intercourse and so treat other female sexual disorders.

Hypoactive sexual desire disorder is present if a woman has no or little desire to be sexual, and has no or few sexual thoughts or fantasies. This type of FSD can be caused by low testosterone levels, due either to natural menopause or to surgical menopause. Other causes include illness, medications, fatigue, depression and anxiety.

Female sexual arousal disorder (FSAD) is characterised by inadequate genital response to sexual stimulation. The genitalia do not undergo the engorgement that characterises normal sexual arousal. The vaginal walls are poorly lubricated, so that intercourse is painful. Orgasms may be impeded. Arousal disorder can be caused by reduced oestrogen at menopause or after childbirth and during lactation, as well as by illnesses, with vascular components such as diabetes and atherosclerosis. Other causes result from treatment with diuretics, antihistamines, antidepressants (e.g. SSRIs) or antihypertensive agents.

Sexual pain disorders (e.g. dyspareunia and vaginismus) is characterised by pain resulting from penetration and may be caused by medications which reduce lubrication, endometriosis, pelvic inflammatory disease, inflammatory bowel disease or urinary tract problems.

The prevalence of FSD is difficult to gauge because the term covers several types of problem, some of which are difficult to measure, and because the interest in treating FSD is relatively recent. Many women's sexual problems are associated either directly with the female ageing process or with chronic illnesses such as diabetes and hypertension.

Because FSD consists of several subtypes that express symptoms in separate phases of the sexual response cycle, there is not a single therapy. Current treatment of FSD focuses principally on psychological or relationship issues. Treatment of FSD is gradually evolving as more clinical and basic science studies are dedicated to the investigation of this medical problem. Female sexual complaints are not all psychological in pathophysiology, especially for those individuals who may have a component of vasculogenic dysfunction (eg FSAD) contributing to the overall female sexual complaint. There are at present no drugs licensed for the treatment of FSD. Empirical drug therapy includes oestrogen administration (topically or as hormone replacement therapy), androgens or mood-altering drugs such as buspirone or trazodone. These treatment options are often unsatisfactory due to low efficacy or unacceptable side effects.

Since interest is relatively recent in treating FSD pharmacologically, therapy consists of the following:—psychological counselling, over-the-counter sexual lubricants, and investigational candidates, including drugs approved for other conditions. These medications consist of hormonal agents, either testosterone or combinations of oestrogen and testosterone and more recently vascular drugs, that have proved effective in male erectile dysfunction. None of these agents has been demonstrated to be very effective in treating FSD.

The Diagnostic and Statistical Manual (DSM) IV of the American Psychiatric Association defines Female Sexual Arousal Disorder (FSAD) as being: “a persistent or recurrent inability to attain or to maintain until completion of the sexual activity adequate lubrication-swelling response of sexual excitement. The disturbance must cause marked distress or interpersonal difficulty.”

The arousal response consists of vasocongestion in the pelvis, vaginal lubrication and expansion and swelling of the external genitalia. The disturbance causes marked distress and/or interpersonal difficulty.

FSAD is a highly prevalent sexual disorder affecting pre-, peri- and post menopausal (±HRT) women. It is associated with concomitant disorders such as depression, cardiovascular diseases, diabetes and UG disorders.

The primary consequences of FSAD are lack of engorgement/swelling, lack of lubrication and lack of pleasurable genital sensation. The secondary consequences of FSAD are reduced sexual desire, pain during intercourse and difficulty in achieving an orgasm.

It has recently been hypothesised that there is a vascular basis for at least a proportion of patients with symptoms of FSAD (Goldstein et al., Int. J. Impot. Res., 10, S84-S90, 1998) with animal data supporting this view (Park et al., Int. J. Impot. Res., 9, 27-37, 1997).

Drug candidates for treating FSAD, which are under investigation for efficacy, are primarily erectile dysfunction therapies that promote circulation to the male genitalia. They consist of two types of formulation, oral or sublingual medications (Apomorphine, Phentolamine, phosphodiesterase type 5 (PDE5) inhibitors e.g. Sildenafil), and prostaglandin (PGE,) that are injected or administered transurethrally in men, and topically to the genitalia in women.

The compounds of the invention are advantageous by providing a means for restoring a normal sexual arousal response—namely increased genital blood flow leading to vaginal, clitoral and labial engorgement. This will result in increased vaginal lubrication via plasma transudation, increased vaginal compliance and increased genital sensitivity. Hence, the compounds of the invention provide means to restore, or potentiate, the normal sexual arousal response.

Without being bound by theory, we believe that neuropeptides such as vasoactive intestinal peptide (VIP) are major neurotransmitter candidates in the control of the female sexual arousal response, especially in the control, of genital blood flow. VIP and other neuropeptides are degraded/metabolised by NEP EC3.4.24.11. Thus, NEP,inhibitors will poteritiate the endogenous vasorelaxant effect of VIP released during arousal. This will lead to a treatment of FSAD, such as through enhanced genital blood flow and hence genital engorgement. We have shown that selective inhibitors of NEP EC 3.4.24.11 enhance pelvic nerve-stimulated and VIP-induced increases in vaginal and clitoral blood flow. In addition, selective NEP inhibitors enhance VIP and nerve-mediated relaxations of isolated vagina wall.

Thus the present invention is advantageous as it helps provide a means for restoring a normal sexual arousal response—namely increased genital blood flow leading to vaginal, clitoral and labial engorgement. This will result in increased vaginal lubrication via plasma transudation, increased vaginal compliance and increased vaginal sensitivity. Hence, the present invention provides a means to restore, or potentiate the normal sexual arousal response.

Male sexual dysfunction includes male erectile dysfunction, ejaculatory disorders such as premature ejaculation (PE), anorgasmia (inability to achieve orgasm) and desire disorders such as hypoactive sexual desire disorder (lack of interest in sex).

It is to be appreciated that all references herein to treatment include curative, palliative and prophylactic treatment.

The compounds of the invention find application in the following sub-populations of patients with FSD: the young, the elderly, pre-menopausal, peri-menopausal, post-menopausal women with or without hormone replacement therapy.

The compounds of the invention find application in patients with FSD arising from:-

i) Vasculogenic etiologies eg cardiovascular or atherosclerotic diseases, hypercholesterolemia, cigarette smoking, diabetes, hypertension, radiation and perineal trauma, traumatic injury to the iliohypogastric pudendal vacular system.

ii) Neurogenic etiologies such as spinal cord injuries or diseases of the central nervous system including multiple sclerosis, diabetes, Parkinsonism, cerebrovascular accidents, peripheral neuropathies, trauma or radical pelvic surgery.

iii) Hormonal/endocrine etiologies such as dysfunction of the hypothalamic/pituitary/gonadal axis, or dysfunction of the ovaries, dysfunction of the pancreas, surgical or medical castration, androgen deficiency, high circulating levels of prolactin eg hyperprolactinemia, natural menopause, premature ovarian failure, hyper and hypothyroidism.

iv) Psychogenic etiologies such as depression, obsessive compulsive disorder, anxiety disorder, postnatal depression/“Baby Blues”, emotional and relational issues, performance anxiety, marital discord, dysfunctional attitudes, sexual phobias, religious inhibition or a traumatic past experiences.

v) Drug-induced sexual dysfunction resulting from therapy with selective serotonin reuptake inhibitors (SSRis) and other antidepressant therapies (tricyclics and major tranquillizers), anti-hypertensive therapies, sympatholytic drugs, chronic oral contraceptive pill therapy.

Patients with mild to moderate MED should benefit from treatment with a compound of the invention and patients with severe MED may also respond. However, early investigations suggest that the responder rate of patients with mild, moderate and severe MED will be greater in combination with a PDE5 inhibitor. Mild, moderate and severe MED will be terms known to the man skilled in the art, but guidance can be found in The Journal of Urology, vol 151, 54-61 (Jan 1994).

The compounds of the invention find application in the following sub-populations of patients with MED: psycogenic, endocrinologic, neurogenic, arteriogenic, drug-induced sexual dysfunction (lactogenic) and sexual dysfunction related to cavernosal factors, particularly venogenic causes. These patient groups are described in more detail in Clinical Andrology vol 23,no.4, p773-782, and chapter 3 of the book by 1. Eardley and K. Sethia “Erectile Dysfunction—Current Investigation and Management, published by Mosby-Wolfe.

Assay Conditions

Production of Native NEP Enzyme:

NEP is isolated from kidneys following the method described by Kenny and Booth (Booth, A. G. & Kenny, A. J. (1974) Biochem. J. 142, 575-581).

Recombinant SEP enzyme is produced using one of two alternative methods:

Method 1: A culture of Chinese Hamster Ovary (CHO) cells is transfected with the plasmid NCIMB deposit number 41110 using the lipofectamine method as described in the lipofectamine reagent protocol (Invitrogen Ltd, Paisley, UK). The cell media is harvested at 24 or 48 hours post transfection, and cleared of cell debris by centrifugation at 3000 g for 5 min. The media is then dialyzed overnight at 4° C. against 50 mM HEPES pH7.4/10% glycerol, using a “slide a lyser” from Pierce and Warner, Chester UK. The dialyzed sample is then frozen in aliquots and stored under liquid nitrogen.

Method 2: A stable human embryonic kidney (HEK) cell line producing recombinant SEP has been made in house according to standard molecular and cell biology methods. This HEK-SEP cell line is cultured in flasks or roller bottles according to standard protocols for HEK cells, in media supplemented with hygromycin B. Media is collected and centrifuged at 3000 g for 15 minutes at room temperature to remove the cell debris, then dialysed with dialysis buffer (50 mM HEPES pH7.4/10% glycerol) for at least 6 hours, using a “slide a lyser” from Pierce and Warner, Chester UK, with at least one change of dialysis buffer during the 6 hours.

Assay of SEP or NEP Peptidase Activity

The peptidase activity of SEP or NEP is measured by monitoring its ability to proteolyse the synthetic peptide substrate Rhodamine green-Gly-Gly-dPhe-Leu-Arg-Arg-Val-Cys(QSY7)-βAla-NH₂:

Reagents for the assay are first prepared as follows:

A substrate solution is made up by diluting a 2 mM/100% DMSO Rhodamine green-Gly-Gly-dPhe-Leu-Arg-Arg-Val-Cys(QSY7)-βAla-NH₂ stock solution in 50 mM HEPES buffer pH7.4 (Sigma, UK) at a concentration of 2 μM.

An aliquot of SEP or NEP enzyme described above is thawed then diluted in 50 mM HEPES, pH7.4 containing 1 EDTA free protease inhibitor cocktail tablet (Roche Diagnostics, UK) per 25 ml. The dilution is by a predetermined factor specific to each enzyme batch, such that 15 μl contains sufficient enzyme to convert approximately 30% of substrate to product during the assay.

A 4% DMSO solution comprised of 4 ml DMSO plus 96 ml 50 mM HEPES pH7.4 is prepared.

A product solution is prepared by adding 500 μl of substrate solution to 250 μl enzyme solution plus 250 μl of 4% DMSO solution, and incubating at 37° C. for 16 hours.

Assays are set up as follows:

In a black 384 well microtitre plate, 15 μl of enzyme solution is added to 15 μl of 4% DMSO solution. A similar non-specific background blank is also set up in which the 15 μl of 4% DMSO solution additionally contains 40 μM phosphoramidon. 30 μl of substrate solution is then added to both the assay and blank, then the plate is incubated for 1 hour at 37° C. Following incubation a fluorescence measurement is taken (Ex485/Em538). BMG galaxy fluorescence reader (BMG Lab technologies, Offenberg, Germany).

The proteolytic activity of the enzyme corresponds to the fluorescence of the sample minus the fluorescence of the non-specific background blank.

A fluorescence measurement taken from 60 μl of product in a well on an identical microtitre plate may be taken. If required this value is used, together with the measured fluorescence units from the SEP assay to calculate the % of the substrate proteolysed during the 1 hour incubation period or to convert the measured fluorescence increase into other useful units such as ng substrate proteolysed/min/ml enzyme.

Using the assay to determine the IC50's of NEP and SEP inhibitors:

To determine the IC₅₀ of SEP or NEP inhibitors (for example phosphoramidon), multiple assays are performed as described above with a range of test concentrations of inhibitor included in the 15 μl of DMSO solution. (Made by appropriate dilution of a 10 mM 100% DMSO stock of inhibitor with 4% DMSO/50 mM HEPES pH7.4.) Using a suitable standard graph fitting computer program, a sigmoidal dose response curve is fitted to a plot of log inhibitor concentration vs % inhibition or % activity. The IC₅₀ is calculated as the inhibitor concentration causing 50% maximal inhibition. Typically for a given IC₅₀ determination, a dose range of at least 10 inhibitor concentrations used differing in half log unit increments is used.

For inhibitors that give an IC₅₀ result less than approximately 2 nM, the assay is repeated under modified assay conditions in which: The quantity of enzyme used is reduced to approx 1/10^{th} to 1/20^{th}; The substrate concentration is increased to 5 μM; and the incubation time increased to 3 hours. This lowers the potency limit (tight binding limit) of the assay to a level where The IC₅₀ estimate of compounds whose Ki is in the range of ˜0.2-2 nM are not limited by the enzyme concentration.

The compounds of the present invention have been tested in the assays above. All the compounds are potent NEP inhibitors with an IC50 of <20 nanomolar and a selectivity for NEP over SEP of at least 1000 fold.

(R)-2-Methyl-3-(1-{[3-(2-methyl-1,3-benzothiazol-6-yl)propyl]carbamoyl}cyclopentyl)propanoic acid (Example 1) has an activity against NEP expressed as an IC50 of 1 nm and 1900 fold selectivity for NEP over SEP.

The utility of the compounds of the present invention to treat FSD and MED may be further determined using the techniques described in WO02/079143.

The advantageous pharmacokinetics of the compounds of the present invention may be demonstrated by using the CaCO-2 test. The CACO-2 assay is a widely accepted model for predicting the ability of a given molecule to cross the GI tract. The compounds of the present invention have good CACO-2 flux defined as follows. Compounds with an apparent permeability (Papp,) value in CACO-2 cells of >5×10⁻⁶ cm/s (at pH 7.4) and >15×10⁻⁶ cm/s (at pH 6.5) are considered to have good permeability and predicted to be well absorbed across the GI tract.

The test is conducted as described below:

Cell Culture

Caco-2 cells were seeded in 24-well Falcon Multiwell® plates at 4.0×10⁴ cells/well. The cells were grown in culture media consisting of minimum essential medium (Gibco 21090-022) supplemented 20% Fetal Bovine serum, 1% non-essential ammino acids, 2 mM L-glutamine and 2 mM sodium pyruvate. The culture medium was replaced three times every week and the cells were maintained at 37° C., with 5% CO₂ and at 90% relative humidity. Permeability studies were conducted when the monolayers were between 15 and 18 days old. Cells were used between passage 23 and 40.

Permeability Studies

Each test compound was prepared as a 1 mM DMSO solution, 62.5 μl of this solution was then added to 25 mL of transport buffer. Nadolol (25 μM) was added to every well as a marker of membrane integrity. These solutions along with transport buffer were then warmed to 37° C. Transport buffer was HBSS (Hank's balanced salt solution) at pH 7.4 or pH 6.5. Before the commencing each study each monolayer was washed three times with HBSS. Transport Buffer with no compound added was placed in each acceptor well, 250 μl on the apical side and 1 mL into the basolateral well. The study was commenced by adding drug solution to each donor well, 250 μl to the apical wells and 1 mL to the basolateral well. Following a two-hour incubation at 37° C. for two hours samples were removed from all wells for LC-MS-MS analysis.

The compounds of the present invention have a CACO-2 A-B flux >5.

Human liver microsomes are a widely accepted model for predicting the metabolic stability of drug molecules towards metabolism in the liver. The compounds of the present invention are stable towards metabolism by HLM. Compounds with a half-life in HLM of <90 mins are metabolised too quickly and are predicted to show a prohibitively short residence time in the body, and reduced bioavailability compared to metabolically stable compounds. The compounds of the present invention have half lifes in HLM of >110 mins.

The test is conducted as follows:

Microsomal Incubations

All incubations were carried out in a thermostatted shaking water-bath at 37° C. Each incubate contained 0.5 μM CYP. Cofactors were added as NADPH regenerating system. It consisted of 1.2 mM NADP, 5 mM MgCl₂×6H₂O, 5 mM DL-isocitric acid and 1 unit/ml isocitric dehydrogenase, highly purified. All reagents were dissolved in phosphate buffer (50 mM; pH 7.4). The substrate concentration was 1 μM. Substrates were dissolved in acetonitrile with the final acetonitrile concentration in the incubation mixture lower than 0.1% (v/v). NADP was omitted from control incubations. In all experiments, samples were pre-incubated with microsomes, substrate and regenerating system in the absence of NADP for 5 min at 37° C. The reaction was started by addition of NADP. Incubation time was 1 h. 100 μl aliquots were removed after 0, 3, 5, 10, 15, 20, 30, 45 & 60 min. The aliquots were extracted with 400 μl 1M-acetic acid and 2.0 ml of ethylacetate and analysed by LC-MS-MS.

The compounds of the may be combined with one or more further active ingredients selected from the list:

1) One or more naturally occurring or synthetic prostaglandins or esters thereof. Suitable prostaglandins for use herein include compounds such as alprostadil, prostaglandin E₁, prostaglandin E₀, 13, 14-dihydroprosta glandin E₁, prostaglandin E₂, eprostinol, natural synthetic and semi-synthetic prostaglandins and derivatives thereof including those described in WO-00033825 and/or U.S. Pat. No. 6,037,346 issued on 14th Mar. 2000 all incorporated herein by reference, PGE₀, PGE₁, PGA₁, PGB₁, PGF₁ α, 19-hydroxy PGA₁, 19-hydroxy -PGB₁, PGE₂, PGB₂, 19-hydroxy-PGA₂, 19-hydroxy-PGB₂, PGE₃α, carboprost tromethamine dinoprost, tromethamine, dinoprostone, lipo prost, gemeprost, metenoprost, sulprostune, tiaprost and moxisylate.

2) One or more α-adrenergic receptor antagonist compounds also known as α-adrenoceptors or α-receptors or α-blockers. Suitable compounds for use herein include: the α-adrenergic receptor blockerss as described in PCT application WO99/30697 published on 14th Jun. 1998, the disclosures of which relating to α-adrenergic receptors are incorporated herein by reference and include, selective α₁-adrenoceptor or α₂-adrenoceptor blockers and non-selective adrenoceptor blockers, suitable α₁-adrenoceptor blockers include: phentolamine, phentolamine mesylate, trazodone, alfuzosin, indoramin, naftopidil, tamsulosin, dapiprazole, phenoxybenzamine, idazoxan, efaraxan, yohimbine, rauwolfa alkaloids, Recordati 15/2739, SNAP 1069, SNAP 5089, RS17053, SL 89.0591, doxazosin, terazosin, abanoquil and prazosin, α₂-blocker blockers from U.S. Pat. No. 6,037,346 [14th Mar. 2000] dibenarnine, tolazoline, trimazosin and dibenarnine; axadrenergic receptors as described in U.S. Pat. Nos.: 4,188,390; 4,026,894; 3,511,836; 4,315,007; 3,527,761; 3,997,666; 2,503,059; 4,703,063; 3,381,009; 4,252,721 and 2,599,000 each of which is incorporated herein by reference; α₂-Adrenoceptor blockers include: clonidine, papaverine, papaverine hydrochloride, optionally in the presence of a cariotonic agent such as pirxamine.

3) One or more NO-donor (NO-agonist) compounds. Suitable NO-donor compounds for use herein include organic nitrates, such as mono- di or tri-nitrates or organic nitrate esters including glyceryl brinitrate (also known as nitroglycerin), isosorbide 5-mononitrate, isosorbide dinitrate, pentaerythritol tetranitrate, erythrityl tetranitrate, sodium nitroprusside (SNP), 3-morpholinosydnonimine molsidomine, S-nitroso-N-acetyl penicilliamine (SNAP) S-nitroso-N-glutathione (SNO-GLU), N-hydroxy-L-arginine, amylnitrate, linsidomine, linsidomine chlorohydrate, (SIN-1) S-nitroso-N-cysteine, diazenium diolates, (NONOates), 1,5-pentanedinitrate, L-arginene, ginseng, zizphi fructus, molsidomine, Re-2047, nitrosylated maxisylyte derivatives such as NMI-678-11 and NMI7-937 as described in published PCT application WO 0012075.

4) One or more potassium channel openers or modulators. Suitable potassium channel openers/modulators for use herein include nicorandil, cromokalim, levcromakalim, lemakalim, pinacidil, cliazoxide, minoxidil, charybdotoxin, glyburide, 4-amini pyridine, BaCl₂.

5) One or more dopaminergic agents, preferably apomorphine or a selective D₂, D₃ or D₂/D₃agonist such as, pramipexole and ropirinol (as claimed in WO-0023056), PNU95666 (as claimed in WO-0040226).

6) One or more vasodilator agents. Suitable vasodilator agents for use, herein include nimodepine, pinacidil, cyclandelate, isoxsuprine, chloroprumazine, halo peridol, Rec 15/2739, trazodone.

7) One or more thromboxane A2 agonists.

8) One or more CNS active agents.

9) One or more ergot alkoloids. Suitable ergot alkaloids are described in U.S. Pat. No. 6,037,346 issued on 14th Mar. 2000 and include acetergamine, brazergoline, bromerguride, cianergoline, delorgotrile, disulergine, ergonovine maleate, ergotamine tartrate, etisulergine, lergotrile, lysergide, mesulergine, metergoline, metergotamine, nicergoline, pergolide, propisergide, proterguride, terguride.

10) One or more compounds which modulate the action of natruretic factors in particular atrial naturetic factor (also known as atrial naturetic peptide), B type and C type naturetic factors such as inhibitors or neutral endopeptidase.

11) One or more compounds which inhibit angiotensin-converting enzyme such as enapril, and combined inhibitors of angiotensin-converting enzyme and neutral endopeptidase such as omapatrilat.

12) One or more angiotensin receptor antagonists such as losartan.

13) One or more substrates for NO-synthase, such as L-arginine.

14) One or more calcium channel blockers such as amlodipine.

15) One or more antagonists of endothelin receptors and inhibitors or endothelin-converting enzyme.

16) One or more cholesterol lowering agents such as statins (e.g. atorvastatin/Lipitor-trademark) and fibrates.

17) One or more antiplatelet and antithrombotic agents, e.g. tPA, uPA, warfarin, hirudin and other thrombin inhibitors, heparin, thromboplastin activating factor inhibitors.

18) One or more insulin sensitising agents such as rezulin and hypqglycaemic agents such as glipizide.

19) L-DOPA or carbidopa.

20) One or more acetylcholinesterase inhibitors such as donezipil.

21) One or more steroidal or non-steroidal anti-inflammatory agents.

22) One or more estrogen receptor modulators and/or estrogen agonists and/or estrogen antagonists, preferably raloxifene, tibolone or lasofoxifene, (−)-cis-6-phenyl-5-[4-(2-pyrrolidin-1-yl-ethoxy)-phenyl]-5,6,7,8-tetrahydronaphthalene-2-ol and pharmaceutically acceptable salts thereof the preparation of which is detailed in WO 96/21656.

23) One or more modulators of cannabinoid receptors.

24) One or more of an NPY (neuropeptide Y) inhibitor, more particularly NPY1 or NPY5 inhibitor, preferably NPY1 inhibitor, preferably said NPY inhibitors (including NPY Y1 and NPY Y5) having an IC50 of less than 100 nM , more preferably less than 50 nM. An assay for identifying NPY inhibitors is presented in WO-A-98/52890 (see page 96, lines 2 to 28).

25) One or more of vasoactive intestinal protein (VIP), VIP mimetic, VIP analogue, more particularly mediated by one or more of the VIP receptor subtypes VPAC1, VPAC or PACAP (pituitory adenylate cyclase activating peptide), one or more of a VIP receptor agonist or a VIP analogue (eg Ro-125-1553) or a VIP fragment, one or more of a α-adrenoceptor antagonist with VIP combination (eg Invicorp, Aviptadil).

26) One or more of a melanocortin receptor agonist or modulator or melanocortin enhancer, such as melanotan II, PT-14, PT-141 or compounds claimed in WO-09964002, WO-00074679, WO-09955679, WO-00105401, WO-00058361, WO-00114879, WO-00113112, WO-09954358.

27) One or more of a serotonin receptor agonist, antagonist or modulator, more particularly agonists, antagonists or modulators for 5HT1A (including VML 670), 5HT2A, 5HT2C, 5HT3 and/or 5HT6 receptors, including those described in WO-09902159, WO-00002550 and/or WO-00028993.

28) one or more of an androgen such as androsterone, dehydro-androsterone, testosterone, androstanedione and a synthetic androgen.

29) one or more of an oestrogen, such as oestradiol, oestrone, oestriol and a synthetic estrogen, such as oestrogen benzoate.

30) One or more of a modulator of transporters for noradrenaline, dopamine and/or serotonin, such as bupropion, GW-320659.

31) One or more of a purinergic receptor agonist and/or modulator.

32) One or more of a neurokinin (NK) receptor antagonist, including those described in WO-09964008.

33) One or more of an opioid receptor agonist, antagonist or modulator, preferably agonists for the ORL-1 receptor.

34) One or more of an agonist or modulator for oxytocin/vasopressin receptors, preferably a selective oxytocin agonist or modulator.

35) One or more of a PDE inhibitor, more particularly a PDE 2, 3, 4, 5, 7 or 8 inhibitor, preferably PDE2 or PDE5 inhibitor and most preferably a PDE5 inhibitor (see hereinafter), said inhibitors preferably having an IC50 against the respective enzyme of less than 100 nM. Suitable cGMP PDE5 inhibitors for the use according to the present invention include:

the pyrazolo [4,3-d]pyrimidin-7-ones disclosed in EP-A-0463756; the pyrazolo [4,3-d]pyrimidin-7-ones disclosed in EP-A-0526004; the pyrazolo [4,3-d]pyrimidin-7-ones disclosed in published international patent application WO 93/06104; the isomeric pyrazolo [3,4-d]pyrimidin-4-ones disclosed in published international patent application WO 93/07149; the quinazolin-4-ones disclosed in published international patent application WO 93/12095; the pyrido [3,2-d]pyrimidin-4-ones disclosed in published international patent application WO 94/05661; the purin-6-ones disclosed in published international patent application WO 94/00453; the pyrazolo [4,3-d]pyrimidin-7-ones disclosed in published international patent application WO 98/49166; the pyrazolo [4,3-d]pyrimidin-7-ones disclosed in published international patent application WO 99/54333; the pyrazolo [4,3-d]pyrimidin-4-ones disclosed in EP-A-0995751; the pyrazolo [4,3-d]pyrimidin-7-ones disclosed in published international patent application WO 00/24745; the pyrazolo [4,3-d]pyrimidin-4-ones disclosed in EP-A-0995750; the compounds disclosed in published international application W095/19978; the compounds disclosed in published international application WO 99/24433 and the compounds disclosed in published international application WO 93/07124. The pyrazolo [4,3-d]pyrimidin-7-ones disclosed in published international application WO 01/27112; the pyrazolo [4,3-d]pyrimidin-7-ones disclosed in published international application WO 01/27113; the compounds disclosed in EP-A-1092718 and the compounds disclose din EP-A-1092719.

Further suitable PDE5 inhibitors for the use according to the present invention include: 5-[2-ethoxy-5-(4-methyl-1-piperazinylsulphonyl)phenyl]-1-methyl-3-n-propyl-1,6-dihydro-7H-pyrazolo[4,3-d]pyrimidin-7-one(sildenafil) also known as 1-[[3-(6,7-dihydro-1-methyl-7-oxo-3-propyl-1H-pyrazolo[4,3-d]pyrimidin-5-yl)-4-ethoxyphenyl]sulphonyl]-4-methylpiperazine (see EP-A-0463756); 5-(2-ethoxy-5-morpholinoacetylphenyl)-1-methyl-3-n-propyl-1,6-dihydro-7H-pyrazolo[4,3-d]pyrimidin-7-one (see EP-A-0526004); 3-ethyl-5-[5-(4-ethylpiperazin-1-ylsulphonyl)-2-n-propoxyphenyl]-2-(pyridin-2-yl)methyl-2,6-dihydro-7H-pyrazolo[4,3-d]pyrimidin-7-one (see WO98/49166); 3-ethyl-5-[5-(4-ethylpiperazin-1-ylsulphonyl)-2-(2-methoxyethoxy)pyridin-3-yl]-2-(pyridin-2-yl)methyl-2,6-dihydro-7H-pyrazolo[4,3-d]pyrimidin-7-one (see WO99/54333); (+)-3-ethyl-5-[5-(4-ethylpiperazin-1-ylsulphonyl)-2-(2-methoxy-1(R)-methylethoxy)pyridin-3-yl]-2-methyl-2,6-dihydro-7H-pyrazolo[4,3-d]pyrimidin-7-one, also known as 3-ethyl-5-{5-[4-ethylpiperazin-1-ylsulphonyl]-2-([(1R)-2-methoxy-1-methylethyl]oxy)pyridin-3-yl}-2-methyl-2,6-dihydro-7H-pyrazolo[4,3-d]pyrimidin-7-one (see WO99/54333); 5-[2-ethoxy-5-(4-ethylpiperazin-1-ylsulphonyl)pyridin-3-yl]-3-ethyl-2-[2-methoxyethyl]-2,6-dihydro-7H-pyrazolo[4,3-d]pyrimidin-7-one, also known as 1-{6-ethoxy-5-[3-ethyl-6,7-dihydro-2-(2-methoxyethyl)-7-oxo-2H-pyrazolo[4,3-d]pyrimidin-5-yl]-3-pyridylsulphonyl}-4-ethylpiperazine (see WO 01/27113, Example 8); 5-[2-iso-Butoxy-5-(4-ethylpiperazin-1-ylsulphonyl)pyridin-3-yl]-3-ethyl-2-(1-methylpiperidin-4-yl)-2,6-dihydro-7H-pyrazolo[4,3-d]pyrimidin-7-one-(see WO 01/27113, Example 15); 5-[2-Ethoxy-5-(4-ethylpiperazin-1-ylsulphonyl)pyridin-3-yl]-3-ethyl-2-phenyl-2,6-dihydro-7H-pyrazolo[4,3-d]pyrimidin-7-one (see WO 01/27113, Example 66); 5-(5-Acetyl-2-propoxy-3-pyridinyl)-3-ethyl-2-(1-isopropyl-3-azetidinyl)-2,6-dihydro-7H-pyrazolo[4,3-d]pyrimidin-7-one (see WO 01/27112, Example 124); 5-(5-Acetyl-2-butoxy-3-pyridinyl)-3-ethyl-2-(1-ethyl-3-azetidinyl)-2,6-dihydro-7H-pyrazolo[4,3-d]pyrimidin-7-one (see WO 01/27112, Example 132); (6R, 12aR)-2,3,6,7,12,12a-hexahydro-2-methyl-6-(3,4-methylenedioxyphenyl)pyrazino[2′,1′:6,1]pyrido[3,4-b]indole-1,4-dione (IC-351), i.e. the compound of examples 78 and 95 of published international application WO95/19978, as well as the compound of examples 1,3,7 and 8; 2-[2-ethoxy-5-(4-ethyl-piperazin-1-yl-1-sulphonyl)-phenyl]-5-methyl-7-propyl-3H-imidazo[5,1-f][1,2,4]triazin-4-one (vardenafil) also known as 1-[[3-(3,4-dihydro-5-methyl-4-oxo-7-propylimidazo[5,1-f]-as-triazin-2-yl)-4-ethoxyphenyl]sulphonyl]-4-ethylpiperazine, i.e. the compound of examples 20, 19, 337 and 336 of published international application WO99/24433; and the compound of example, 11 of published international application WO93/07124 (EISAI); and compounds 3 and 14 from Rotella D P, J. Med. Chem., 2000, 43, 1257.

Still other suitable PDE5 inhibitors include:4-bromo-5-(pyridylmethylamino)-6-[3-(4-chlorophenyl)-propoxy]-3(2H )pyridazinone; 1-[4-[(1,3-benzodioxol-5-ylmethyl)amiono]-6-chloro-2-quinozolinyl]-4-piperidine-carboxylic acid, monosodium salt; (+)-cis-5,6a,7,9,9,9a-hexahydro-2-[4-(trifluoromethyl)-phenylmethyl-5-methyl-cyclopent-4,5]imidazo[2,1-b]purin-4(3H)one; furazlocillin; cis-2-hexyl-5-methyl-3,4,5,6a,7,8,9,9a- octahydrocyclopent[4,5]-imidazo[2,1-b]purin-4-one; 3-acetyl-1-(2-chlorobenzyl)-2-propylindole-6-carboxylate; 3-acetyl-1-(2-chlorobenzyl)-2-propylindole-6-carboxylate; 4-bromo-5-(3-pyridylmethylamino)-6-(3-(4-chlorophenyl) propoxy)-3-(2H)pyridazinone; I-methyl-5(5-morpholinoacetyl-2-n-propoxyphenyl)-3-n-propyl-1,6-dihydro-7H-pyrazolo(4,3-d)pyrimidin-7-one; 1-[4-[(1,3-benzodioxol-5-ylmethyl)amino]-6-chloro-2-quinazolinyl]-4-piperidinecarboxylic acid, monosodium salt; Pharmaprojects No. 4516 (Glaxo Wellcome); Pharmaprojects No. 5051 (Bayer); Pharmaprojects No. 5064 (Kyowa Hakko; see WO 96/26940); Pharmaprojects No. 5069 (Schering Plough); GF-196960 (Glaxo Wellcome); E-8010 and E-4010 (Eisai); Bay-38-3045 & 38-9456 (Bayer) and Sch-51866.

For treating FSD, the compounds of the invention may preferably be combined with one or more active ingredients selected from the list:

a) a PDE5 inhibitor, more preferably 5-[2-ethoxy-5-(4-methyl-1-piperazinylsulphonyl)phenyl]-1-methyl-3-n-propyl-1,6-dihydro-7H-pyrazolo[4,3-d]pyrimidin-7-one (sildenafil); (6R, 12aR)-2,3,6,7,12,12a-hexahydro-2-methyl-6-(3,4-methylenedioxyphenyl)-pyrazino[2′,1′:6,1]pyrido[3,4-b]indole-1,4-dione (IC-351); 2-[2-ethoxy-5-(4-ethyl-piperazin-1-yl-1-sulphonyl)-phenyl]-5-methyl-7-propyl-3H-imidazo[5,1-f][1,2,4]triazin-4-one (vardenafil); 5-[2-ethoxy-5-(4-ethylpiperazin-1-ylsulphonyl)pyridin-3-yl]-3-ethyl-2-[2-methoxyethyl]-2,6-dihydro-7H-pyrazolo[4,3-d]pyrimidin-7-one; and 5-(5-acetyl-2-butoxy-3-pyridinyl)-3-ethyl-2-(1-ethyl-3-azetidinyl)-2,6-dihydro-7H-pyrazolo[4,3-d]pyrimidin-7-one and pharmaceutically acceptable salts thereof;

b) an NPY Y1 inhibitor;

c) a dopamine agonist such as apomorphine or a selective D₂, D₃ or D₂/D₃ agonist such as, pramipexole and ropirinol;

d) a melanocortin receptor agonist or modulator or melanocortin enhancer, preferably melanotan II, PT-14, PT-141;

e) an agonist, antagonist or modulator for 5HT2C;

f) an estrogen receptor modulator, estrogen agonists and/or estrogen antagonists, preferably raloxifene, tibolone or lasofoxifene;

g) an androgen such as androsterone, dehydro-androsterone, testosterone, androstanedione and a synthetic androgen; and

h) an oestrogen, such as oestradiol, oestrone, oestriol and a synthetic estrogen, such as oestrogen benzoate.

For treating MED, the compounds of the invention may preferably be combined with one or more active ingredients selected from the list:

a) a PDE5 inhibitor, more preferably 5-[2-ethoxy-5-(4-methyl-1-piperazinylsulphonyl)phenyl]-1-methyl-3-n-propyl-1,6-dihydro-7H-pyrazolo[4,3-d]pyrimidin-7-one (sildenafil); (6R, 12aR)-2,3,6,7,12,12a-hexahydro-2-methyl-6-(3,4-methylenedioxyphenyl)-pyrazino[2′,1′:6,1]pyrido[3,4-b]indole-1,4-dione (IC-351); 2-[2-ethoxy-5-(4-ethyl-piperazin-1-yl-1-sulphonyl)-phenyl]-5-methyl-7-propyl-3H-imidazo[5,1-f][1,2,4]triazin-4-one (vardenafil); 5-[2-ethoxy-5-(4-ethylpiperazin-1-ylsulphonyl)pyridin-3-yl]-3-ethyl-2-[2-methoxyethyl]-2,6-dihydro-7H-pyrazolo[4,3-d]pyrimidin-7-one; and 5-(5-acetyl-2-butoxy-3-pyridinyl)-3-ethyl-2-(1-ethyl-3-azetidinyl)-2,6-dihydro-7H-pyrazolo[4,3-d]pyrimidin-7-one and pharmaceutically acceptable salts thereof;

b) an NPY Y1 inhibitor;

c) a dopamine agonist (preferably apomorphine) or a selective D₂, D₃ or D₂/D₃ agonist such as, pramipexole and ropirinol;

d) a melanocortin receptor agonist or modulator or melanocortin enhancer, preferably melanotan II, PT-14, PT-141; and

e) an agonist, antagonist or modulator for 5HT2C;

Particularly preferred combinations for treating FSD are the compounds of the present invention and one or more active ingredients selected from the list:

5-[2-ethoxy-5-(4-methyl-1-piperazinylsulphonyl)phenyl]-1-methyl-3-n-propyl-1,6-dihydro-7H-pyrazolo[4,3-d]pyrimidin-7-one (sildenafil);

(6R, 12aR)-2,3,6,7,12,12a-hexahydro-2-methyl-6-(3,4-methylenedioxyphenyl)-pyrazino[2′,1′:6,1]pyrido[3,4-b]indole-1,4-dione (IC-351);

2-[2-ethoxy-5-(4-ethyl-piperazin-1-yl-1-sulphonyl)-phenyl]-5-methyl-7-propyl-3H-imidazo[5,1-f][1,2,4]triazin-4-one (vardenafil);

5-[2-ethoxy-5-(4-ethylpiperazin-1-ylsulphonyl)pyridin-3-yl]-3-ethyl-2-[2-methoxyethyl]-2,6-dihydro-7H-pyrazolo[4,3-d]pyrimidin-7-one;

5-(5-acetyl-2-butoxy-3-pyridinyl)-3-ethyl-2-(1-ethyl-3-azetidinyl)-2,6-dihydro-7H-pyrazolo[4,3-d]pyrimidin-7-one;

apomorphine;

melanotan II;

PT-141;

lasofoxifene;

raloxifene;

tibolone;

an androgen such as androsterone, dehydro-androsterone, testosterone, androstanedione and a synthetic androgen; and

an oestrogen, such as oestradiol, oestrone, oestriol and a synthetic estrogen, such as oestrogen benzoate.

Particularly preferred combinations for treating MED are the compounds of the present invention and one or more active ingredients selected from the list:

5-[2-ethoxy-5-(4-methyl-1-piperazinylsulphonyl)phenyl]-1-methyl-3-n-propyl-1,6-dihydro-7H-pyrazolo[4,3-d]pyrimidin-7-one (sildenafil);

(6R, 12aR)-2,3,6,7,12,12a-hexahydro-2-methyl-6-(3,4-methylenedioxyphenyl)-pyrazino[2′,1′:6,1]pyrido[3,4-b]indole-1,4-dione (IC-351);

2-[2-ethoxy-5-(4-ethyl-piperazin-1-yl-1-sulphonyl)-phenyl]-5-methyl-7-propyl-3H-imidazo[5,1-f][1,2,4]triazin-4-one (vardenafil);

5-[2-ethoxy-5-(4-ethylpiperazin-1-ylsulphonyl)pyridin-3-yl]-3-ethyl-2-[2-methoxyethyl]-2,6-dihydro-7H-pyrazolo[4,3-d]pyrimidin-7-one;

5-(5-acetyl-2-butoxy-3-pyridinyl)-3-ethyl-2-(1-ethyl-3-azetidinyl)-2,6-dihydro-7H-pyrazolo[4,3-d]pyrimidin-7-one;

apomorphine;

melanotan II; and

PT-141.

For treating cardiovascular disorders, particular hypertension, the compounds of the invention may be combined with one or more active ingredient selected from the list:

a) angiotensin receptor blockers (ARB), such as losartan, valsartan, telmisartan, candesartan, irbesartan, eprosartan and olmesartan;

b) calcium channel blockers (CCB) such as amlodipine;

c) statins, such as atorvastatin;

d) PDE5 inhibitors, such as sildenafil, tadalafil, vardenafil, 5-[2-ethoxy-5-(4-ethylpiperazin-1-ylsulphonyl)pyridin-3-yl]-3-ethyl-2-[2-methoxyethyl]-2,6-dihydro-7H-pyrazolo[4,3-d]pyrimidin-7-one; 5-(5-acetyl-2-butoxy-3-pyridinyl)-3-ethyl-2-(1-ethyl-3-azetidinyl)-2,6-dihydro-7H-pyrazolo[4,3-d]pyrimidin-7-one and; the pyrazolo[4,3-d]pyrimidin-4-ones disclosed in WO00/27848 particularly N-[[3-(4,7-dihydro-1-methyl-7-oxo-3-propyl-1H-pyrazolo[4,3-d]-pyrimidin-5-yl)-4-propxyphenyl]sulfonyl]-1-methyl2-pyrrolidinepropanamide [DA-8159 (Example 68 of WO00/27848)];

e) beta blockers, such as atenolol or carvedilol;

f) ACE inhibitors, such as quinapril, enalapril and lisinopril;

g) alpha-blockers such as doxazosin;

h) selective aldosterone receptor antagonists (SARA), such as eplerenone or spironolactone; and

i) imidazoline I₁ agonists, such as rilmenidine and moxonidine.

If a combination of active agents are administered, then they may be administered simultaneously, separately or sequentially.

The compounds of the invention can be administered alone but, in human therapy will generally be administered in admixture with a suitable pharmaceutical excipient diluent or carrier selected with regard to the intended route of administration and standard pharmaceutical practice.

The present invention provides a pharmaceutical composition comprising a compound of formula (I) or pharmaceutically acceptable salts, solvates or polymorphs thereof, and a pharmaceutically acceptable diluent or carrier.

Oral Administration

The compounds of the invention may be administered orally. Oral administration may involve swallowing, so that the compound enters the gastrointestinal tract, or buccal or sublingual administration may be employed by which the compound enters the blood stream directly from the mouth.

Formulations suitable for oral administration include solid formulations such as tablets, capsules containing particulates, liquids, or powders, lozenges (including liquid-filled), chews, multi- and nano-particulates, gels, films (including muco-adhesive), ovules, sprays and liquid formulations.

Liquid formulations include suspensions, solutions, syrups and elixirs. Such formulations may be employed as fillers in soft or hard capsules and typically comprise a carrier, for example, water, ethanol, propylene glycol, methylcellulose, or a suitable oil, and one or more emulsifying agents and/or suspending agents. Liquid formulations may also be prepared by the reconstitution of a solid, for example, from a sachet.

The compounds of the invention may also be used in fast-dissolving, fast-disintegrating dosage forms such as those described in Expert Opinion in Therapeutic Patents, 11 (6), 981-986 by Liang and Chen (2001).

A typical tablet may be prepared using standard processes known to a formulation chemist, for example, by direct compression, granulation (dry, wet, or melt), melt congealing, or extrusion. The tablet formulation may comprise one or more layers and may be coated or uncoated.

Examples of excipients suitable for oral administration include carriers, for example, cellulose, calcium carbonate, dibasic calcium phosphate, mannitol and sodium citrate, granulation binders, for example, polyvinylpyrrolidine, hydroxypropylcellulose, hydroxypropylmethylcellulose and gelatin, disintegrants, for example, sodium starch glycolate and silicates, lubricating agents, for example, magnesium stearate and stearic acid, wetting agents, for example, sodium lauryl sulphate, preservatives, anti-oxidants, flavours and colourants.

Solid formulations for oral administration may be formulated to be immediate and/or modified release. Modified release formulations include delayed-, sustained-, pulsed-, controlled dual-, targeted and programmed release. Details of suitable modified release technologies such as high energy dispersions, osmotic and coated particles are to be found in Verma et al, Pharmaceutical Technology On-line, 25(2), 1-14 (2001). Other modified release formulations are described in U.S. Pat. No. 6,106,864.

Parenteral Administration

The compounds of the invention may also be administered directly into the blood stream, into muscle, or into an internal organ. Suitable means for parenteral administration include intravenous, intraarterial, intraperitoneal, intrathecal, intraventricular, intraurethral, intrasternal, intracranial, intramuscular and subcutaneous. Suitable devices for parenteral administration include needle (including microneedle) injectors, needle-free injectors and infusion techniques.

Parenteral formulations are typically aqueous solutions which may contain excipients such as salts, carbohydrates and buffering agents (preferably to a pH of from 3 to 9), but, for some applications, they may be more suitably formulated as a sterile non-aqueous solution or as a dried form to be used in conjunction with a suitable vehicle such as sterile, pyrogen-free water.

The preparation of parenteral formulations, under sterile conditions, for example, by lyophilisation, may readily be accomplished using standard pharmaceutical techniques well known to those skilled in the art.

The solubility of compounds of formula (I) used in the preparation of parenteral solutions may be increased by suitable processing, for example, the use of high energy spray-dried dispersions (see WO 01/47495) and/or by the use of appropriate formulation techniques, such as the use of solubility-enhancing agents.

Formulations for parenteral administration may be formulated to be immediate and/or modified release. Modified release formulations include delayed-, sustained-, pulsed-, controlled dual-, targeted and programmed release.

Topical Administration

The compounds of the invention may also be administered topically to the skin (preferably to the genitalia) or mucosa, either dermally or transdermally. Typical formulations for this purpose include gels, hydrogels, lotions, solutions, creams, ointments, dusting powders, dressings, foams, films, skin patches, wafers, implants, sponges, fibres, bandages and microemulsions. Liposomes may also be used. Typical carriers include alcohol, water, mineral oil, liquid petrolatum, white petrolatum, glycerin and propylene glycol. Penetration enhancers may be incorporated—see, for example, Finnin and Morgan, J Pharm Sci, 88 (10), 955-958 (October 1999). Other means of topical administration include delivery by iontophoresis, electroporation, phonophoresis, sonophoresis and needle-free or microneedle injection.

Formulations for topical administration may be formulated to be immediate and/or modified release. Modified release formulations include delayed-, sustained-, pulsed-, controlled dual-, targeted and programmed release. Thus compounds of the invention may be formulated in a more solid form for administration as an implanted depot providing long-term release of the active compound.

Inhaled/Intranasal Administration

The compounds of the invention can also be administered intranasally or by inhalation, typically in the form of a dry powder (either alone, as a mixture, for example, in a dry blend with lactose, or as a mixed component particle, for example, mixed with phospholipids) from a dry powder inhaler or as an aerosol spray from a pressurised container, pump, spray, atomiser (preferably an atomiser using electrohydrodynamics to produce a fine mist), or nebuliser, with or without the use of a suitable propellant, such as dichlorbfluoromethane.

The pressurised container, pump, spray, atomizer, or nebuliser contains a solution or suspension of the active compound comprising, for example, ethanol (optionally, aqueous ethanol) or a suitable alternative agent for dispersing, solubilising, or extending release of the active, the propellant(s) as solvent and an optional surfactant, such as sorbitan trioleate or an oligolactic acid.

Prior to use in a dry powder or suspension formulation, the drug product is micronised to a size suitable for delivery by inhalation (typically less than 5 microns). This may be achieved by any appropriate comminuting method, such as spiral jet milling, fluid bed jet milling, supercritical fluid processing to form nanoparticles, high pressure homogenisotion, or spray drying.

A suitable solution formulation for use in an atomiser using electrohydrodynamics to produce a fine mist may contain from 1 μg to 10 mg of the compound of the invention per actuation and the actuation volume may vary from 1 μl to 100 μl. A typical formulation may comprise a compound of formula (I), propylene glycol, sterile water, ethanol and sodium chloride. Alternative solvents which may be used instead of propylene glycol include glycerol and polyethylene glycol.

Capsules, blisters and cartridges (made, for example, from gelatin or HPMC) for use in an inhaler or insufflator may be formulated to contain a powder mix of the compound of the invention, a suitable powder base such as lactose or starch and a performance modifier such as /-leucine, mannitol, or magnesium stearate.

In the case of dry powder inhalers and aerosols, the dosage unit is determined by means of a valve which delivers a metered amount. Units in accordance with the invention are typically arranged to administer a metered dose or “puff” containing from 1 μg to 50 mg of the compound of formula (I). The overall daily dose will typically be in the range 1 μg to 50 mg, such as 1 mg to 50 mg, which may be administered in a single dose or, more usually, as divided doses throughout the day.

Formulations for inhaled/intranasal administration may be formulated to be immediate and/or modified release. Modified release formulations include delayed-, sustained-, pulsed-, controlled dual-, targeted and programmed release.

Rectal/Intravaginal Administration

The compounds of the invention may be administered rectally or vaginally, for example, in the form of a suppository, pessary, or enema. Cocoa butter is a traditional suppository base, but various alternatives may be used as appropriate.

Formulations for rectal/vaginal administration may be formulated to be immediate and/or modified release. Modified release formulations include delayed-, sustained-, pulsed-, controlled dual-, targeted and programmed release.

Ocular/Andial Administration

The compounds of the invention may also be administered directly to the eye or ear, typically in the form of drops of a micronised suspension or solution in isotonic, pH-adjusted, sterile saline. Other formulations suitable for ocular and andial administration include ointments, biodegradable (e.g. absorbable gel sponges, collagen) and non-biodegradable (e.g. silicone) implants, wafers, lenses and particulate or vesicular systems, such as niosomes or liposomes. A polymer such as crossed-linked polyacrylic acid, polyvinylalcohol, hyaluronic acid, a cellulosic polymer, for example, hydroxypropylmethylcellulose, hydroxyethylcellulose, or methyl cellulose, or a heteropolysaccharide polymer, for example, gelan gum, may be incorporated together with a preservative, such as benzalkonium chloride. Such formulations may also be delivered by iontophoresis.

Formulations for ocular/andial administration may be formulated to be immediate and/or modified release. Modified release formulations include delayed-, sustained-, pulsed-, controlled dual-, targeted, or programmed release.

Enabling Technologies

The compounds of the invention may be combined with soluble macromolecular entities such as cyclodextrin or polyethylene glycol-containing polymers to improve their solubility, dissolution rate, taste-masking, bioavailability and/or stability.

Drug-cyclodextrin complexes, for example, are found to be generally useful for most dosage forms and administration routes. Both inclusion and non-inclusion complexes may be used. As an alternative to direct complexation with the drug, the cyclodextrin may be used as an auxiliary additive, i.e. as a carrier, diluent, or solubiliser. Most commonly used for these purposes are alpha-, beta- and gamma-cyclodextrins, examples of which may be found in International Patent Applications Nos. WO 91/11172, WO 94/02518 and WO 98/55148.

Dosage

For administration to human patients, the total daily dose of the compounds of the invention is typically in the range 0.1 mg to 1000 mg depending, of course, on the mode of administration. For example, oral administration may require a total daily dose of from 5 mg to 1000 mg, such as from 5 to 500 mg, while an intravenous dose may only require from 0.01 to 30 mg/kg body weight, such as from 0.1 to 10 mg/kg, more preferably from 0.1 to 1 mg/kg body weight. The total daily dose may be administered in single or divided doses.

These dosages are based on an average human subject having a weight of about 65 to 70 kg. The physician will readily be able to determine doses for subjects whose weight fails outside this range, such as infants and the elderly.

The skilled person will also appreciate that, in the treatment of certain conditions (including FSD and MED), compounds of the invention may be taken as a single dose on an “as required” basis (i.e. as needed or desired).

In a preferred embodiment, the compounds of the invention are delivered systemically (such as orally, buccally and sublingually), more preferably orally. Preferably such systemic (most preferably oral) administration is used to treat female sexual dysfunction, preferably FSAD.

Thus in a particularly preferred embodiment, there is provided the use of the compounds of the invention in the manufacture of a systemically delivered (preferably orally delivered) medicament for the treatment or prophylaxis of FSD, more preferably FSAD.

A preferred oral formulation uses immediate release tablets; or fast dispersing or dissolving dosage formulations (FDDFs).

In a further preferred embodiment, the compounds of the invention are administered topically, preferably directly to the female genitalia, especially the vagina.

Since NEP is present throughout the body, it is very unexpected that the compounds of the invention can be administered systemically and achieve a therapeutic response in the female genitalia without provoking intolerable (adverse) side effects. In EP 1 097 719-A1 and the animal model hereinafter, we have shown that NEP inhibitors administered to a rabbit model (in vivo) systemically increased genital blood flow, upon sexual arousal (mimicked by pelvic nerve stimulation) without adversely affecting cardiovascular parameters, such as causing a significant hypotensive or hypertensive.

Preferably the compounds of the invention are administered for the treatment of FSD in the sexually stimulated patient (by sexual stimulation we mean to include visual, auditory or tactile stimulation). The stimulation can be before, after or during said administration.

Thus the compounds of the invention enhance the pathways/mechanisms that underlie sexual arousal in the female genitalia restoring or improving the sexual arousal response to sexual stimulation.

Thus a preferred embodiment provides the use of a compound of the invention in the preparation of a medicament for the treatment or prophylaxis of FSD in the stimulated patient.

For veterinary use, a compound of the invention is administered as a suitably acceptable formulation in accordance with normal veterinary practice and the veterinary surgeon will determine the dosing regimen and route of administration which will be most appropriate for a particular animal.

The following formulation examples are illustrative only and are not intended to limit the scope of the invention. “Active ingredient” means a compound of the invention.

Formulation 1: A Tablet is Prepared Using the Following Ingredients:

| | |
|---|---|
| Active ingredient | 250 |
| Cellulose, microcrystalline | 400 |
| Silicon dioxide, fumed | 10 |
| Stearic acid | 5 |
| Total | 665 |

The components are blended and compressed to form tablets.

Formulation 2: An Intravenous Formulation May be Prepared as Follows:

| | |
|---|---|
| Active ingredient | 100 mg |
| Isotonic saline | 1,000 ml |

Typical formulations useful for administering the compounds of the invention topically to the genitalia are as follows:

Formulation 3: A Spray

Active ingredient (1.0%) in isopropanol (30%) and water.

Formulation 4: A Foam

Active ingredient, acetic acid glacial, benzoic acid, cetyl alcohol, methyl parahydroxybenzoate, phosphoric acid, polyvinyl alcohol, propylene glycol, sodium carboxymethylcellulose, stearic acid, diethyl stearamide, van Dyke perfume No. 6301, purified water and isobutane.

Formulation 5: A Gel

Active ingredient, docusate sodium BP, isopropyl alcohol BP, propylene glycol, sodium hydroxide, carbomer 934P, benzoic acid and purified water.

Formulation 6: A Cream

Active ingredient, benzoic acid, cetyl alcohol, lavender, compound 13091, methylparaben, propylparaben, propylene glycol, sodium carboxymethylcellulose, sodium lauryl sulfate, stearic acid, triethanolmine, acetic acid glacial, castor oil, potassium hydroxide, sorbic acid and purified water.

Formulation 7: A Pessary

Active ingredient, cetomacrogol 1000 BP, citric acid, PEG 1500 and 1000 and purified water.

The invention additionally includes:

(i) A pharmaceutical composition including a compound of the invention, together with a pharmaceutically acceptable excipient, diluent or carrier.

(ii) A compound of the invention or a pharmaceutically acceptable salt, solvate or polymorph thereof, for use as a medicament.

(iii) The use of a compound of the invention as a medicament for treating or preventing a condition for which a beneficial therapeutic response can be obtained by the inhibition of neutral endopeptidase.

(iv) The use of a compound of the invention as a medicament for treating or preventing hypoactive sexual desire disorder, sexual arousal disorder, orgasmic disorder or sexual pain disorder, preferably sexual arousal disorder, orgasmic disorder or sexual pain disorder, more preferably sexual arousal disorder.

(v) A method of treating FSD or MED in a, mammal including treating said mammal with an effective amount of a compound of the invention.

(vi) An FSD or MED treating pharmaceutical composition comprising a compound of the invention together with a pharmaceutically acceptable excipient, diluent or carrier.

(vii) A compound of the invention for use in treating FSD or MED.

(viii) The use of a compound of the invention in the manufacture of a medicament for treating or preventing FSD or MED.

The invention is illustrated by the following non-limiting examples in which the following abbreviations and definitions are used:

Arbocel® filter agent

br broad

brm broad multiplet

Boc tert-butoxycarbonyl

CDI 1,1′-carbonyldiimidazole

δ chemical shift

d doublet

Δ heat

DCCI dicyclohexylcarbodiimide

DCM dichloromethane

DMA dimethylacetamide

DMF N,N-dimethylformamide

DMSO dimethylsulfoxide

ES⁺ electrospray ionisation positive scan

ES⁻ electrospray ionisation negative scan

Ex Example

h hours

HOBt 1-hydroxybenzotriazole

HPLC high pressure liquid chromatography

m multiplet

m/z mass spectrum peak

min minutes

m.p. Melting point

MiBK Methyl iso-butyl ketone

MS mass spectrum

NMR nuclear magnetic resonance

Prec precursor

Prep preparation

q quartet

quin quintet

s singlet

t triplet

t-BMA tert-butylmethyl ether

Tf trifluoromethanesulfonyl

TFA trifluoroacetic acid

THF tetrahydrofuran

TLC thin layer chromatography

TS⁺ thermospray ionisation positive scan

WSCDI 1-(3-dimethylaminopropyl)-3-ethylcarbodiimide hydrochloride

¹H Nuclear magnetic resonance (NMR) spectra were in all cases consistent with the proposed structures. Characteristic chemical shifts (δ) are given in parts-per-million downfield from tetramethylsilane using conventional abbreviations for designation of major peaks: e.g. s, singlet; d, doublet; t, triplet; q, quartet; m, multiplet; br, broad. The following abbreviations have been used for common solvents: CDCl₃, deuterochloroform; DMSO, dimethylsulphoxide. The abbreviation psi means pounds per square inch and LRMS means low resolution mass spectrometry. Where thin layer chromatography (TLC) has been used it refers to silica gel TLC using silica gel 60 F₂₅₄ plates, R_{f} is the distance travelled by a compound divided by the distance travelled by the solvent front on a TLC plate. Melting points were determined using a Perkin Elmer DSC7 at a heating rate of 20° C./minute).

### EXAMPLE 1

### (R)-2-Methyl-3-(1-{[3-(2-methyl-1,3-benzothiazol-6-yl)propyl]carbamoyl}cyclopentyl)propanoic acid

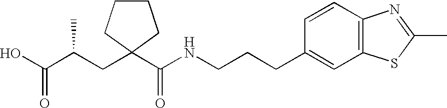

tert-Butyl (2R)-2-methyl-3-[1-({[3-(2-methyl-1,3-benzothiazol-6-yl)propyl]-amino}carbonyl)cyclopentyl]propanoate (Preparation 7 and Preparation 8) (7.4 g, 16.7 mmol) was dissolved in dichloromethane (10 mL) and trifluoroacetic acid (10 mL) was added and the mixture stirred at room temperature for 5 hours. The reaction was quenched by the addition of potassium carbonate (10% aqueous solution) to adjust the pH to ca. 3 (ca. 120 mL required). The resulting mixture was extracted with dichloromethane (3×100 mL) and the combined organic layers dried with MgSO₄ and evaporated. The residue was purified by flash chromatography [SiO₂; methanol in dichloromethane 1% to 2%] to afford the desired acid as a clear oil (5.66 g, 87%). This batch was combined with 1.4 g of material from a previous run and stirred in pentane (100 mL) for 3 hours. The pentane layer was removed, the residue scratched to loosen up the gummy residue and stirred with a further portion of pentane (100 mL) for a further 2 hours. The resulting white powder was collected on a sinter funnel and dried under vacuum at 45° C. to afford the title compound as a free flowing white powder (m.p. 105-106° C.) (6.52 g). Found; C, 64.93; H, 7.29; N, 7.18. C₂₁H₂₈N₂O₃S requires C, 64.92; H, 7.26; N, 7.21. ¹H NMR (CDCl₃, 500 MHz) δ_{H}: 7.85 (1H, d), 7.61 (1H, d), 7.23 (1H, dd), 5.89 (1H, brm), 3.25-3.35 (2H, m), 2.81 (3H, s), 2.74 (2H, m), 2.45 (1H, m), 2.10 (1H, m), 1.98 (1H, m), 1.89 (1H, m), 1.88 (2H, m), 1.55-1.68 (5H, m), 1.49 (2H, m) 1.17 (3H, d). ¹³C NMR (CDCl₃, 125 MHz) δ_{C}: 180.5, 177.4, 166.7, 151.7, 138.5, 135.9, 126.7, 122.1, 120.7, 54.5, 42.7, 39.6, 37.4, 36.6, 36.1, 33.4, 31.3, 24.0, 24.3, 19.9, 19.3 m/z (electrospray negative ion) 387 (M-H⁺)

Optical rotation measurements were taken in methanol solution (5.7 mg, in 5 mL) with the following results:

| | |
|---|---|
| [α]₅₈₉²⁵ | −4.4 |
| [α]₅₇₈²⁵ | −4.6 |
| [α]₅₄₆²⁵ | −5.4 |
| [α]₄₈₉²⁵ | −7.9 |
| [α]₃₆₅²⁵ | −13.0 |

Purity was assessed as >99% by HPLC analysis using five different reverse phase columns:

| | Percentage Purity | | | | |
|---|---|---|---|---|---|
| | 225 nm | | 254 nm | | main peak |
| | Main | Main | Main | Main | retention |
| | Peak | Impurity | Peak | Impurity | time/min |
| Phenomenex | 100% | — | 99.95% | 0.05% | 3.8 |
| Phenyl Hexyl | | | | | |
| 3 μm | | | | | |
| Phenomenex | 99.95% | 0.05% | 99.9% | 0.04% | 3.8 |
| Synergi Polar | | | | | |
| RP 4 μm | | | | | |
| Develosil | 100% | — | 99.9% | 0.04% | 3.9 |
| Combi RP C30 | | | | | |
| 3 μm | | | | | |
| Dionex Acclaim | 99.95% | 0.05% | 99.9% | 0.03% | 3.9 |
| C18 3 μm | | | | | |
| Gravity C18 | 99.4% | 0.06% | 99.9% | 0.04% | 3.6 |
| 3 μm | | | | | |

| | |
|---|---|
| HPLC conditions | Analytical |
| Temperature | Ambient |
| Detection | 225, 254 nm |
| Mobile phase | A: Water:MeCN:TFA |
| | 95:5:0.1% (v/v) |
| | B: MeCN |
| | Linear gradient elution (see below) |
| Flow rate | 1 ml/min |

Solvent gradient conditions:

| Time | % B |
|---|---|
| 0.0 | 0 |
| 0.2 | 0 |
| 5.0 | 95 |
| 7.1 | 95 |
| 8.0 | 0 |

Chiral purity was assessed as 98% by capillary electrophoresis by comparison to an authentic sample of the opposite enantiomer prepared by a similar route, using the conditions described below:

| CE conditions | |
|---|---|
| Capillary | Agilent fused silica extended light path capillary |
| | 64.5 cm (56 cm effective length), 50 μm I.D. |
| Temperature | 15° C. |
| Detection | UV at 230, 254 and 260 nm |
| Sample | c.a.1 mg/ml in run buffer:water:methanol:acetone; |
| dissolution | (1:10:1:0.5) |
| System/data | HP 3DCE (see attached printouts) |
| file | |
| Injection | 4 seconds 50 mbar sample then 2 seconds 50 mbar water |
| Run buffer | 250 mg α-cyclodextin and 50 mg SBE-β-cyclodextrin |
| | dissolved in pH 9.3 borax buffer, 50 mM (Agilent CE |
| | solution), 5 ml. |
| Pre- | New capillary: 10 minutes 930 mBar 1.0 M NaOH |
| conditioning | Between runs: 2 minutes 930 mbar 0.1 M NaOH, rinse |
| | 2 sec water, 4 minutes 930 mBar run buffer |
| Voltage | 25 kV (ramped 0-25 kV over 30 seconds) |
| Run Time | 20 minutes |

An alternative procedure for the production of the title compound is given below:

tert-Butyl (2R)-2-methyl-3-[1-({[3-(2-methyl-1,3-benzothiazol-6-yl)propyl]-amino}carbonyl)cyclopentyl]propanoate (Preparation 7 and Preparation 8) (2.4 g, 5.4 mmol) was dissolved in toluene (7 ml) and trifluoroacetic acid (4.1 ml) was added and the mixture stirred at 17° C. for 6 hours. The reaction was quenched by the addition of sodium carbonate (9% aqueous solution) to adjust the pH to 3 (30 ml required). MiBK (15 ml) was added. The organic phase was separated and the product extracted into sodium carbonate (9% aqueous solution, 2×5 ml). The product was extracted into isopropyl acetate (35 ml) by pH adjustment with 5M HCl to pH 4.5 over 1 hour. The organic phase was concentrated to 5 ml/g with respect to starting material by atmospheric distillation. The oil was cooled to ambient temperature, crystallised and granulated at 0 to −5° C. for one hour. The solid was collected using vacuum filtration, washed with isopropyl acetate (5 ml) and dried under vacuum at 40° C. overnight to afford the title compound as a free flowing white powder (m.p. 105-106° C.) [1.3 g, 3.3 mmol, (62%)].

### EXAMPLE 2

### 3-(1-{[3-(2-ethyl-1,3-benzothiazol-6-yl)propyl]carbamoyl}cyclopentyl)propanoic acid

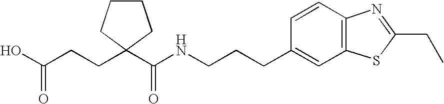

This compound was prepared using a procedure analogous to that described in Example 1, starting from tert-butyl 3-[1-({[3-(2-ethyl-1,3-benzothiazol-6-yl)propyl]amino}carbonyl)-cyclopentyl]propanoate from preparation 9.

m.p. 127.5-129.5° C.; ¹H NMR (d₆-DMSO, 400 MHz) δ_{H}: 7.89 (1H, d), 7.63 (1H, s), 7.25 (1H, d), 5.69 (1H, brm), 3.37 (2H, m), 3.15 (2H, q), 2.76 (2H, m), 2.31 (2H, m), 1.96-1.87 (6H, m), 1.70-1.55 (4H, m), 1.47 (3H, t); m/z (ES⁺) 411 (MNa⁺), 389 (MH⁺); m/z (ES⁻) 387 (M-H⁺). Found; C, 64.74; H, 7.28; N, 7.14. C₂₁H₂₈N₂O₃S requires C, 64.92; H, 7.26; N; 7.21.

Purity was assessed as >98% by HPLC analysis using three different reverse phase columns:

| | Percentage Purity | | | | |
|---|---|---|---|---|---|
| | 225 nm | | 254 nm | | main peak |
| | Main | Main | Main | Main | retention |
| | Peak | Impurity | Peak | Impurity | time/min |
| Luna Phenyl | 99.25% | 0.51% | 98.79% | 0.56% | 9.80 |
| Hexyl 3 μm | | | | | |
| (150 × 4.6 mm) | | | | | |
| Phenomenex | 99.25% | 0.49% | 99.14% | 0.44% | 9.98 |
| Synergi Polar | | | | | |
| RP 4 μm (150 × | | | | | |
| 4.6 mm) | | | | | |
| Curosil PFP | 99.31% | 0.4% | 98.21% | 0.84% | 9.05 |
| (150 × 4.6 mm) | | | | | |

| | |
|---|---|
| HPLC conditions | Analytical |
| Temperature | Ambient |
| Detection | 225, 254 nm |
| Mobile phase | A: Water:MeCN:TFA |
| | 95:5:0.1% (v/v) |
| | B: MeCN |
| | gradient elution (see below) |
| Flow rate | 1 ml/min |

Solvent gradient conditions:

| Time (min) | % B |
|---|---|
| 0.2-15 | 0-100 |
| 15-18 | 100 |
| 18-18.2 | 100-0 |
| 18.2-20 | 0 |

### EXAMPLE 3

### 3-(1-{[3-(2-ethyl-1,3-benzothiazol-6-yl)propyl]carbamoyl}cyclohexyl)propanoic acid

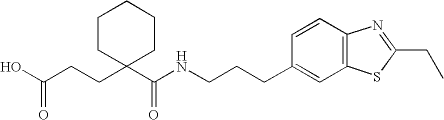

This compound was prepared using a procedure analogous to that described in Example 1, starting from tert-Butyl 3-(1-{[3-(2-ethyl-1,3-benzothiazol-6-yl)propyl]carbamoyl}cyclohexyl)-propanoate from preparation 12.

¹H NMR (d₆-DMSO, 400 MHz) δ_{H}: 7.86 (1H, d), 7.64 (1H, d), 7.24 (1H, dd), 5.72.(1H, brs), 3.32 (2H, m), 3.13 (2H, q), 2.75 (2H, m), 2.27 (2H, m), 1.88 (2H, quin), 1.85-1.73 (4H, m), 1.60-1.23 (8H, m), 1:46 (3H, t); m/z (ES⁺) 425 (MNa⁺), 403 (MH⁺); m/z (ES⁻) 401 (M-H⁺)

### EXAMPLE 4

### (R)-2-Methyl-3-(1-{[3-(2-ethyl-1,3-benzothiazol-6-yl)propyl]carbamoyl}cyclopentyl)propanoic acid

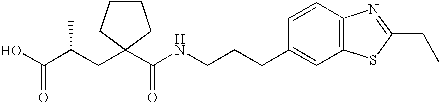

The title compound was prepared using the procedure described in Example 1, starting from tert-Butyl (2R)-2-methyl-3-[1-({[3-(2-ethyl-1,3-benzothiazol-6-yl)propyl]amino}carbonyl) cyclopentyl]propanoate from preparation 10.

¹H NMR (CDCl₃, 400 MHz) δ_{H}: 7.86 (1H, d), 7.62 (1H, d), 7.24 (1H, dd), 5.81 (1H, brm), 3.27 (2H, m), 3.12 (2H, q), 2.74 (2H, m), 2.43 (1H, m), 2.11-2.05 (1H, m), 1.98-1.81 (4H, m), 1.69-1.40 (7H, m), 1.45 (3H, t), 1.16 (3H, d); m/z (ES⁺) 425 (MNa⁺), 403 (MH⁺); m/z (ES⁻) 401 (M-H⁺)

### EXAMPLE 5

### 3-(1-{[3-(2-methyl-1,3-benzothiazol-6-yl)propyl]carbamoyl}cyclohexyl)propanoic acid

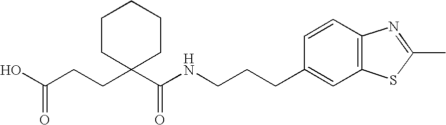

The title compound was prepared using a procedure analogous to that described in Example 1, starting from tert-Butyl 3-(1-{[3-(2-methyl-1,3-benzothiazol-6-yl)propyl]carbamoyl}cyclohexyl)-propanoate from preparation 13.

¹H NMR (CD₃OD, 400 MHz) δ_{H}: 7.78 (1H, d), 7.71 (1H, brm), 7.34 (1H, d), 3.29-3.23 (obscured) (2H, m), 2.80 (3H, s), 2.77 (2H, m), 2.22-2.17 (2H, m), 2.05-1.98 (2H, m), 1.92-1.85 (2H, m), 1.78-1.74 (2H, m), 1.63-1.51 (3H, m), 1.44-1.22 (5H, m); m/z (APCl⁺) 389 (MH⁺). Found; C, 64.05; H, 7.39; N, 8.82. C₂₁H₂₈N₂O₃S.0.25H₂O requires C, 64.18; H, 7.31; N, 7.13.

### EXAMPLE 6

### 3-(1-{[3-(2-methyl-1,3-benzothiazol-6-yl)propyl]carbamoyl}cyclopentyl)propanoic acid

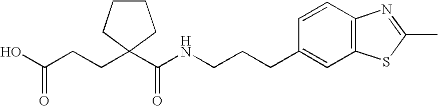

The title compound, was prepared using a procedure analogous to that described in. Example 1, starting from. tert-Butyl 3-(1-{[3-(2-methyl-1,3-benzothiazol-6-yl)propyl]carbamoyl}cyclopentyl)propanoate from preparation 14.

¹H NMR (CDCl₃, 400 MHz) δ_{H}: 7.85 (¹H, d), 7.61 (1H, d), 7.24 (1H, dd), 5.70 (1H, brs), 3.30 (1H, m), 2.81 (3H, s), 2.74 (2H, m), 2.30 (2H, m), 1.97-1.84 (6H, m), 1.69-1.56 (4H, m), 1.50-1.41 (2H, m); m/z (APCl⁺) 375 (MH⁺); (APCl⁻) 373 (MH⁺)

### Preparation 1

### Di(tert-butyl)3-(2-methyl-1,3-benzothiazol-6-yl)propylimidodicarbonate

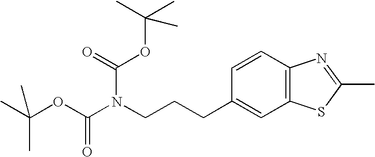

Di (tert-butyl) allylimidodicarbonate [Bioorganic & Medicinal Chemistry Letters, 1999, 7, (1625-1636] (8.75 g, 34 mmol) was treated with 9-BBN [Aldrich] (136 mL, 0.5M solution in tetrahydrofuran, 2 equivalents, 68 mmol) at 0° C., and the solution stirred at room temperature for 45 min. Potassium phosphate (23 mL, 3M aqueous solution, 2.0 equivalents, 69 mmol) was added cautiously and the reaction flask then covered with aluminium foil. A solution of 6-bromo-2-methylbenzothiazole [J. Chem. Soc. 1936-1225; DE3528032A1] (7.80 g, 34 mmol, 1 equivalent) in dimethylformamide (50 mL) was added followed by 1,1′-bis(diphenylphosphino)ferrocene palladium(II) dichloride-dichloromethane 1:1 complex) (2.77 g, 3.4 mmol, 0.1 equivalents) and the reaction mixture stirred at room temperature for 18 hours. The reaction mixture was concentrated in vacuo and the residue purified by column chromatography [SiO₂, pentane: ethyl acetate, 5:1 then 3:1] to afford the desired product as a clear oil (11.2 g, 84%). ¹H NMR analysis showed the material showed slight contamination with traces of solvent and 9-BBN residues. ¹H NMR (CDCl₃, 400 MHz) δ_{H}: 7.81 (1H, d), 7.60 (1H, s), 7.23 (1H, d), 3.60 (2H, t), 2.77 (3H, s), 2.71 (2H, t), 1.97-1.88 (2H, m), 1.45 (18H, s): m/z (APCl⁺) 407 (MH⁺), 307 (MH⁺-Boc)

### Preparation 2

### Di(tert-butyl)3-(2-ethyl-1,3-benzothiazol-6-yl)propylimidodicarbonate

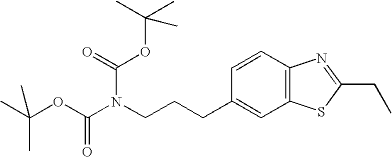

This 2-ethylthiazole intermediate was prepared in an analogous fashion that of preparation 1 using 6-bromo-2-ethylbenzothiazole [Bull. Soc. Chim. Fr. 1967, 2812-23] as the aryl bromide component. ¹H NMR (d₆-DMSO, 400 MHz) δ_{H}: 7.84 (1H, d), 7.64 (1H, s), 7.26 (1H, d), 3.60 (2H, m), 3.12 (2H, q), 2.73 (2H, t), 1.07-1.86 (2H, m), 1.47 (9H, s), 1.45 (3H, t): m/z (APCl⁺) 421 (MH⁺), 321 (MH⁺-Boc)

### Preparation 3

### 3-(2-methyl-1,3-benzothiazol-6-yl)propylamine dihydrochloride salt

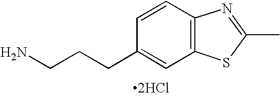

N,N-di-tert-butoxycarbonyl-3-(2-methyl-1,3-benzothiazol-6-yl)propylamine (11.2 g, 27.5 mmol) was dissolved in 1,4-dioxane (30 mL) and treated with hydrogen chloride (25 mL, 4M solution in 1,4-dioxane, 100 mmol) and the solution stirred at room temperature for 18 hours. The reaction mixture was evaporated to a white solid which was triturated with a mixture of pentane and diethyl ether (3:1) to afford the desired amine bis-HCl salt as a white solid (6.05 g, 78%). ¹H NMR (d₆-DMSO, 400 MHz) δ_{H}: 7.98 (3H, brs), 7.85 (1H, s), 7.81 (1H, d), 7.31 (1H, d), 2.78-2.73 (4H, m), 2.75 (3H, s), 1.94-1.85 (2H, m): m/z (APCl⁺) 207.

Alternative procedure for the production of the title compound are given in preparations 4 and 5 below.

### Preparation 4

### 3-(2-methyl-1,3-benzothiazol-6-yl)propylamine dihydrochloride salt (Alternative Procedure)

Step (1):

3-(2-Methyl-benzothiazol-6-yl)-acrylonitrile

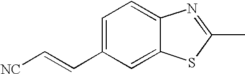

To a stirred solution of 6-iodo-2-methyl-benzothiazole [WO2002090443A1] (13.1 g, 47.64 mmol) in DMF (500 mL) under N₂ at room temperature was added acrylonitrile (6.27 mL, 95.28 mmol), then NaOAc (2.86 g, 47.04 mmol), then Pd(OAc)₂ (1.07 g, 4.76 mmol), and then P(o-tolyl)₃ (2.90 g, 9.53 mmol). The resulting mixture was stirred under N₂ at 130° C. for ˜24hrs then at room temperature for 2 days. Quenched with water (750 mL) then extracted (Et₂O, 3×200 mL). The combined organic layers were dried (MgSO₄), filtered, and concentrated in vacuo. The resulting residue was purified by flash column chromatography eluting with 10:1 pentane:EtOAc then 3:1 pentane:EtoAc to afford the title compound as an oil (9.00 g, 94%) and as a ˜3:1 mixture of trans:cis isomers. ¹H NMR (δ_{H}) 2.85 (3H, m), 5.45 and 5.90 (1H, m), 7.50 (2H, m), 7.90 (2H, m); APCl-MS 201 (100%) [MH⁺].

Step (2):

[3-(2-Methyl-benzothiazol-6-yl)-propyl]-carbamic acid tert-butyl ester

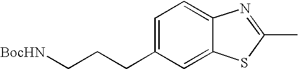

To a stirred solution of 3-(2-methyl-benzothiazol-6-yl)-acrylonitrile (9.00 g, 45.00 mmol) in MeOH (1L) under N₂ at room temperature was added Boc₂O (19.64 mL, 90.00 mmol) and then NiCl₂ (5.84 g, 45.00 mmol). To the resulting mixture was added portionwise over ˜20 mins with care NaBH₄ (13.62 g, 300.00 mmol). The resulting mixture was stirred under N₂ at room temperature for ˜1.5 hrs the filtered through Arbocel and concentrated in vacuo. The resulting residue was purified by flash column chromatography eluting with 3:1 pentane:EtOAc then 1:1 pentane:EtoAc to afford the title compound as an oil (5.00 g, 36%). ¹H NMR (δ_{H}) 1.40 (9H, s), 1.85 (1H, m), 2.65 (1H, m), 2.75 (1H, m), 2.90 (3H, m), 3.05 (1H, m), 3.15 (1H, m), 3.50 (2H, m), 7.30 (1H, m), 7.65 (1H, m), 7.90 (1H, m); APCl-MS 307 (75%) [MH⁺], 251 (50%), 203 (100%).

Step (3):

3-(2-Methyl-benzothiazol-6-yl)-propylamine dihydrochloride salt

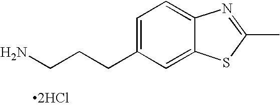

To a stirred solution of [3-(2-methyl-benzothiazol-6-yl)-propyl]-carbamic acid tert-butyl ester (248 mg, 0.78 mmol) in DCM (10 mL) under N₂ at room temperature was added 4N HCl in dioxan (5 mL) and the resulting mixture was stirred under N₂ at room temperature for 4 hrs. The solvents were removed in vacuo and the residue triturated with Et₂O (3×10 mL). The filtrate was concentrated in vacuo to afford the title compound as an colourless foam (195 mg, 99%) judged by ¹H NMR to be of sufficient purity to be used in the next stage without further.

### Preparation 5

### 3-(2-methyl-1,3-benzothiazol-6-yl)propylamine dihydrochloride salt (Alternative Procedure)

Step(1)

2-[3-(2-methyl-benzothiazol-5-yl)-propyl]-isoindole-1,3-dione

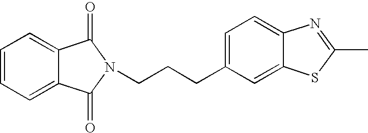

To a slurry of 2-(2-propenyl)-1H-Isoindole-1,3(2H)-dione (18.7 g, 0.1 mol) (allyl phthalimide: Journal of Organic Chemistry (1952), 17 68-76). in THF (38 ml, 2 ml/g) was added a solution (0.5 M) of 9-BBN in THF (240, 1.2 eq) over 45 minutes keeping the temperature between 0° C. and 5° C. The solution was then warmed to ambient over one hour and stirred for a further one hour. A solution of K₂CO₃ (27.6 g, 0.20 mol, 2 eq) in 50 ml water was added over 15 minutes, followed by 2-methyl-6-bromo-benzothiazole (20.5 g,. 0.09 mol, 0.9 eq.) dissolved in DMF (120 ml ) and Dichloro [1,1′-bis(diphenylphosphino)ferrocene]palladium (II) dichloromethane adduct (2.4 g , 0.03 eq). The reaction was warmed to 50° C. over 1 hour and then immediately cooled to ambient temperature. Water (260 ml, 19 ml/g) and t-BME (560 ml, 30 ml/g) were added and the solution stirred. The reaction was filtered through filter aid to remove any particulates. A phase separation was performed and the upper organic phase retained. The organic phase was concentrated by vacuum filtration to approximately 3 ml/g with respect to theoretical product. The dark slurry was granulated at 0° C. to −5° C. for 30 minutes, filtered under vacuum and then washed with chilled t-BME (0.4 L, 4.5 ml/g). The resulting solid was dried under vacuum at 55° C. overnight to give 2-[3-(2-methyl-benzothiazol-5-yl)-propyl]-isoindole-1,3-dione (20.5 g, 0.061 mol, 68%).

m.p. 126° C.; ¹H-NMR (d₆-DMSO , 300 MHz), δ: 1.95-1.98 (m, 2 H), 2.72-2.77 (m, 5 H), 3.63 (t, 2 H), 7.32 (d, 1 H), 7.75 (d, 1 H), 7.74-7.83 (m, 5 H).

Step (2)

3-(2-methyl-1,3-benzothiazol-6-yl)propylamine dihydrochloride salt:

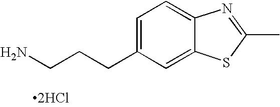

To a slurry of 2-[3-(2-methyl-benzothiazol-5-yl)-propyl]-isoindole-1,3-dione (20 g, 0.06 mol) in water (130 ml, 6.5 ml/g) and ethyl alcohol (200 ml, 10 ml/g) was added aqueous methylamine (40% w/w solution, 117 g, 1.54 mol, 26 eq) over 10 minutes to form a solution. The solution was stirred for 18 hours at 25° C. Dichloromethane (300 ml, 15 ml/g) was added and the reaction stirred for 15 minutes. A phase separation was performed and the organic product phase retained. Residual product was extracted from the aqueous phase with dichloromethane (100 ml, 5 ml/g). The combined organic phases were washed with 1M K₂CO₃ (300 ml, 15 ml/g). A phase separation was performed and the organic phase washed with water (300 ml, 15 ml/g). The organic phase was stripped to a low volume under vacuum and exchanged with iso propyl alcohol to give a final reaction volume of 10 ml/g with respect to 2-[3-(2-methyl-benzothiazol-5-yl)-propyl]-isoindole-1,3-dione. The isopropyl alcohol solution was warmed to 70° C. and concentrated HCl (12.5 ml, 0.125 mol, 2.1 eq) added over 10 minutes. IPA (90 ml, 4.5 ml/g) was removed by distillation at atmospheric pressure. The slurry was cooled the reaction solution to 25° C., granulated at 0-−50° C. for 1 hour, filtered under vacuum and washed with chilled IPA (40 ml, 2 ml/g). The product was dried under vacuum at 55° C. over night to give 3-(2-methyl-1,3-benzothiazol-6-yl)propylamine dihydrochloride salt [12.0 g, 0.058 mol (72%)]; m.p. 215° C.

### Preparation 6

### 3-(2-ethyl-1,3-benzothiazol-6-yl)propylamine dihydrochloride salt

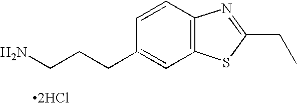

This 2-ethylthiazole intermediate was prepared in an analogous fashion that of preparation 3, except that dichloromethane was used as the initial solvent in place of dioxane.

¹H NMR (CDCl₃, 400 MHz) δ_{H}: 8.04 (1H, s), 7.95 (1H, d), 7.62 (1H, d), 3.36 (2H, q), 3.02-2.88 (4H, m), 2.10-2.02 (2H, m), 1.53 (3H, t): m/z 221 (MH⁺)

### Preparation 7

### tert-Butyl (2R)-2-methyl-3-[1-({[3-(2-methyl-1,3-benzothiazol-6-yl)propyl]-amino}carbonyl)cyclopentyl]propanoate

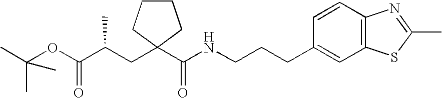

A solution of 1-[(2R)-3-tert-butoxy-2-methyl-3-oxopropyl]cyclopentanecarboxylic acid [WO0279143A1] (6.8 g, 26.5 mmol) in isopropyl acetate (30 mL) was added to a solution of 1,1′-carbonyldiimidazole (4.76 g, 29.3 mmol, 1.1 equivalents) in isopropyl acetate (60 mL) and the mixture heated for 3 hours at 60° C. and then overnight at room temperature. An aliquot was removed and evaporated to dryness and tested by ¹H NMR spectroscopy. This indicated the reaction has proceeded to approximately 90% conversion (Me doublet for starting acid δ 1.15, Me doublet for acyl imidazolide δ 1.05). A further portion of 1,1′-carbonyldiimidazole (645 mg, 4 mmol, 0.15 equivalents) was added and the mixture stirred at 60° C. for a further 1 hour. The reaction mixture was cooled to 40° C. and triethylamine (4.3 mL, 31 mmol, 1.1 equivalents) and 3-(2-methyl-1,3-benzothiazol-6-yl)propylamine dihydrochloride salt from preparation 3, 4 or 5 (7.1 g, 25.4 mmol, 0.96 equivalents) were added and the mixture heated at 60° C. overnight. Thin layer chromatography showed the reaction was not complete, therefore a further portion of amine dihydrochloride was added (650 mg, 2.3 mmol, 0.09 equivalents) together with triethylamine (330 μL, 2.3 mmol, 0.09 equivalents) and the mixture heated at 60° C. overnight. The reaction mixture was allowed to cool to room temperature and then diluted with water (120 mL) and 2M hydrochloric acid (120 mL). The mixture was extracted with diethyl ether (2×400 mL) and the combined extracts washed with 2M NaOH (2×100 mL), dried (MgSO₄) and evaporated. The oily residue obtained was purified by flash chromatography [SiO₂, methanol in dichloromethane 0.5% to 1.5%] to afford the title compound as a clear oil (11.5 g, 98%). ¹H NMR (CDCl₃, 500 MHz) δ_{H}: 7.85 (1H, d), 7.63 (1H, d), 7.27 (1H dd), 5.77 (1H, brs), 3.35-3.25 (2H, m), 2.82 (3H, s), 2.77 (2H, m), 2.33 (1H, m), 2.03-2.03 (2H, m), 1.89 (3H, m), 1.68-1.55 (5H, m), 1.45-1.44 (2H, s), 1.42 (9H, s) 1.10 (3H, d). ¹³C NMR (CDCl₃, 125 MHz) δ_{C}: 176.6, 176.6, 166.5, 151.5, 135.8, 126.9, 122.1, 120.8, 80.2, 54.6, 42.4, 39.3, 38.4, 37.3, 35.0, 33.4, 31.6, 28.0, 24.3, 23.8, 20.0, 19.6. m/z (ES⁺) 467 MNa⁺.

An alternative procedure for the title compound is given in preparation 8 below:

### Preparation 8

### tert-Butyl (2R)-2-methyl-3-[1-({[3-(2-methyl-1,3-benzothiazol-6-yl)propyl]-amino}carbonyl)cyclopentyl]propanoate (Alternative Procedure)

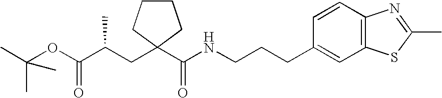

To a slurry of 1,1′-carbonyldiimidazole (154.6 g, 1.02 mols) in isopropyl acetate (2.5 ml/g) maintained at 60° C. was added a solution of 1-[(2R)-3-tert-butoxy-2-methyl-3-oxopropyl]cyclopentanecarboxylic acid [WO0279143A1] (238.4 g, 0.93 mol) in isopropyl acetate (2.2 ml/g) under a blanket of nitrogen gas over 2 hours. Residual 1-[(2R)-3-tert-butoxy-2-methyl-3-oxopropyl]cyclopentanecarboxylic acid solution was washed in with isopropyl acetate (0.3 ml/g). The solution was stirred and maintained at 60° C. for a further 5 hours and then cooled to room temperature 3-(2-methyl-1,3-benzothiazol-6-yl)propylamine dihydrochloride salt from preparation 3, 4 or 5 (259.8 g, 0.93 mols) was added to the solution. Triethylamine (188.3 g, 1.86 mols) was added over 30 minutes. The mixture was heated to reflux over 10 minutes and then maintained at reflux for 4 hours, and then cooled to room temperature. The mixture was filtered to remove insoluble material and washed with isopropyl acetate (2.0 ml/g). The wash was combined with the filtrate. Water (2.0 ml/g) was added and the mixture acidified to pH 5.0 by adding 5M hydrochloric acid (375 ml). The mixture was filtered to remove insoluble material. A phase separation was performed and the upper organic phase retained. This Was then washed with 0.5 M aqueous potassium carbonate solution (238 ml), a phase separation was performed and the upper organic phase retained. This was washed with saturated brine solution until the pH was less than 9.0. This solution was diluted with isopropyl acetate to 5 ml/g and then distilled and replaced with toluene at atmospheric pressure maintaining constant volume. For analytical purposes a sample could be evaporated to dryness to give the product as an oil (389 g, 0.87 mol, 94% yield).

### Preparation 9

### tert-butyl 3-[1-({[3-(2-ethyl-1,3-benzothiazol-6-yl)propyl]amino}carbonyl)cyclopentyl]propanoate

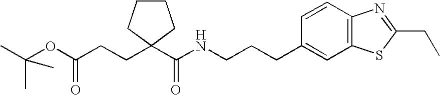

1-(3-tert-butoxy-3-oxopropyl)cyclopentanecarboxylic acid [WO0279143A1] (180 mg, 0.7 mmol) was mixed together with the 3-(2-ethyl-1,3-benzothiazol-6-yl)propylamine dihydrochloride salt from preparation 6 (180 mg, 0.65 mmol), 1-(3-dimethylaminopropyl)-3-ethylcarbodiimide hydrochloride (135 mg, 0.7 mmol), 1-hydroxybenzotriazole (95 mg, 0.7 mmol) and triethylamine (400 μL, 2.8 mmol) in dimethyl formamide (7 mL). The reaction mixture was stirred at 60° C. overnight. After cooling to room temperature the mixture was evaporated in vacuo and the residue diluted with water (50 mL). The mixture was extracted with diethyl ether (3×50 mL) and the combined organic fractions dried with MgSO₄ and then evaporated. Purification by flash chromatography [SiO₂; ethyl acetate in pentane (15% to 20%)] afforded the desired compound as a light brown oil. ¹H NMR (CDCl₃, 400 MHz) δ_{H}: 7.87 (1H, d), 7.65 (1H, d), 7.26 (1H, dd), 5.63 (1H, brs), 3.30 (2H, q), 3.12 (2H, q), 2.75 (2H, t), 2.20-2.14 (2H, m), 1.98-1.80 (6H, m), 1.68-1.58 (5H, m), 1.45 (3H, t), 1.42 (9H, s): m/z (ES⁺) 467 (MNa⁺), 445 (MH⁺).

Alternatively, the title compound was made using a method analogous to that described in preparation 7.

### Preparation 10

### tert-Butyl (2R)-2-methyl-3-[1-({[3-(2-ethyl-1,3-benzothiazol-6-yl)propyl]amino}carbonyl) cyclopentyl]propanoate

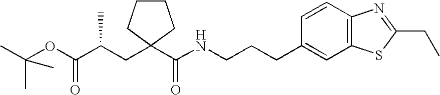

The title compound was prepared using a method analogous to that described for preparation 9 by coupling 3-(2-ethyl-1,3-benzothiazol-6-yl)propylamine dihydrochloride salt from preparation 6 together with 1-[(2R)-3-tert-butoxy-2-methyl-3-oxopropyl]cyclopentanecarboxylic acid [W00279143A1].

¹H NMR (CDCl₃, 400 MHz) δ_{H}: 7.87 (1H, d), 7.66 (1H, s), 7.27 (1H, m), 5.77 (1H, brm), 3.29 (2H, m), 3.13 (2H, q), 2.77 (2H, m), 2.31 (1H, m), 2.08-1.96 (2H, m), 1.70-1.43 (10H, m), 1.45 (3H, t), 1.43 (9H, s), 1.10 (3H, d): m/z (ES⁺) 481 (MNa⁺), 459 (MH⁺)

### Preparation 11

### 1-(2-tert-Butoxycarbonyl-ethyl)-cyclohexanecarboxylic acid

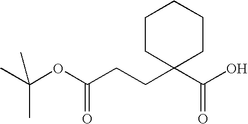

Cyclohexanecarboxylic acid (2.89 g, 22.6 mmol) was dissolved in THF (30 mL) and this solution added to a lithium diisopropylamide solution cooled to −15° C. (2M in THF/n-heptane/ethylbenzene, Aldrich) 24.3 mL (48.6 mmol) at such a rate as to keep the temperature below 0° C. The reaction mixture was then stirred at 0° C. for 2.5 hours before re-cooling to −15° C. and the addition of tert-butyl 3-bromopropionate (5 g, 23.9 mmol) in THF (50 mL) at such a rate as to keep the temperature below 0° C. The reaction mixture was allowed to reach room temperature and stirred overnight, before the reaction mixture was quenched by the addition of 2M HCl and extracted with ethyl. acetate 2×200 mL. The combined organic layers were dried (MgSO₄) and then evaporated to an orange oil. Purification by flash chromatography [SiO₂; dichloromethane/methanol/0.880 NH₃ (97:3:0.5)] afforded the title compound as a gummy oil (800 mg, 14%). ¹H NMR (CDCl₃, 400 MHz), δ_{H}: 2.22 (2H, m), 2.07-2.00 (2H, m), 1.86 (2H, m), 1.45-1.16 (8H,m), 1.41 (9H, s): m/z (ES⁺) 279(MNa⁺); m/z (ES⁻) 255 M−H⁺)

### Preparation 12

### tert-Butyl 3-(1-{[3-(2-ethyl-1,3-benzothiazol-6-yl)propyl]carbamoyl}cyclohexyl)propanoate

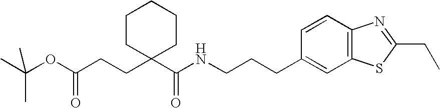

The title compound was prepared by a method analogous to that described for preparation 9 using 3-(2-ethyl-1,3-benzothiazol-6-yl)propylamine dihydrochloride salt from preparation 6 together with 1-[(2R)-3-tert-butoxy-2-methyl-3-oxopropyl]cyclohexanecarboxylic acid from preparation 11.

¹H NMR (CDCl₃, 400 MHz) δ_{H}: 7.88 (1H, d), 7.65 (1H, s), 7.27 (1H, m), 5.65 (1H, brm), 3.34 (2H, m), 3.14 (2H, q), 2.77 (2H, m), 2.15 (1H, m), 1.95-1.27 (14H, m), 1.45 (3H, t), 1.41 (9H, s): m/z (ES⁺) 481 (MNa⁺), 459 (MH⁺)

### Preparation 13

### tert-Butyl 3-(1-{[3-(2-methyl-1,3-benzothiazol-6-yl)propyl]carbamoyl}cyclohexyl)propanoate

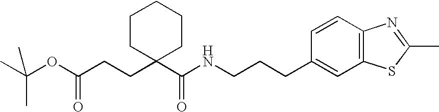

The title compound was prepared by a method analogous to that described for preparation 9 using 3-(2-methyl-1,3-benzothiazol-6-yl)propylamine dihydrochloride salt from preparation 3, 4 or 5 together with 1-[(2R)-3-tert-butoxy-2-methyl-3-oxopropyl]cyclohexanecarboxylic acid from preparation 11.

¹H NMR (CDCl₃, 400 MHz) δ_{H}: 7.85 (1H, d), 7.64 (1H, d), 7.27 (obscured) (1H, dd), 5.65 (1H, brm), 3.33 (2H, m), 2.81 (3H, s), 2.77 (2H, m), 2.18-2.13 (2H, m), 1.93-1.79 (4H, m), 1.74-1.68 (2H, m), 1.60-1.23 (7H, m), 1.40 (9H,s): m/z (ES⁺) 467 (MNa⁺), 445 (MH⁺)

### Preparation 14

### tert-Butyl 3-(1-{[3-(2-methyl-1,3-benzothiazol-6-yl)propyl]carbamoyl}cyclopentyl)propanoate

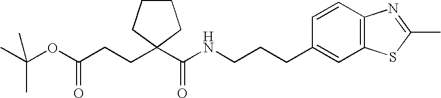

The title compound was prepared using a method analogous to that described for preparation 9 by coupling 3-(2-methyl-1,3-benzothiazol-6-yl)propylamine dihydrochloride salt from preparation 3, 4 or 5 together with tert-Butyl-3-1-carboxycyclopentyl)proponoate [WO0279143A1]

¹H NMR (CDCl₃, 400 MHz) δ_{H}: 7.85 (1H, d), 7.62 (1H, d), 7.25 (1H,m), 5.63 (1H, brm), 3.29 (2H, m), 2.81 (3H, s), 2.75 (2H, m), 2.19-2.15 (2H,m), 2.00-1.36 (11H, m), 1.42 (9H, s); m/z (APCl⁺) 431 (MH⁺).

1. A compound of formula (I) 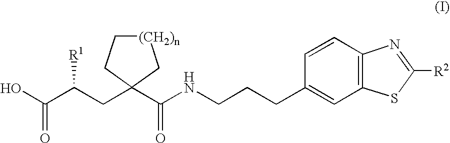
wherein:
R¹ is H or CH₃;
R² is C₁-C₂ alkyl; and
n is 1 or 2;
a tautomer thereof or a pharmaceutically acceptable salt, or solvate of said compound or tautomer.

2. The compound according to claim 1 wherein n is 1.

3. The compound according to claim 1 or claim 2 wherein R¹ is hydrogen.

4. The compound according to claim 3 wherein R² is methyl.

5. The compound according to claim 3 wherein R² is ethyl.

6. The compound according claim 1 or claim 2 wherein R¹ is methyl.

7. The compound according to claim 6 wherein R² is methyl.

8. The compound according to claim 6 wherein R² is ethyl.

9. The compound according to claims 1 or 2 wherein R² is methyl.

10. The compound according to claims 1 or 2 wherein R² is ethyl.

11. The compound according to claim 1 selected from
(R)-2-Methyl-3-(1-{[3-(2-methyl-1,3-benzothiazol-6-yl)propyl]carbamoyl}cyclopentyl)propanoic acid;
3-(1-{[3-(2-ethyl-1,3-benzothiazol-6-yl)propyl]carbamoyl}cyclopentyl)propanoic acid;
(R)-2-Methyl-3-(1-{[3-(2-ethyl-1,3-benzothiazol-6-yl)propyl]carbamoyl}cyclopentyl)propanoic acid; or
3-(1-{[3-(2-ethyl-1,3-benzothiazol-6-yl)propyl]carbamoyl}cyclohexyl)propanoic acid.

12. A compound of Formula Ia, 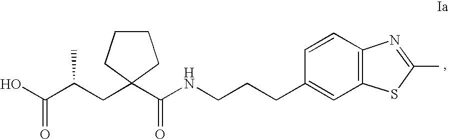
a tautomer thereof or a pharmaceutically acceptable salt, or solvate of said compound or tautomer.

13. A pharmaceutical composition comprising a compound of formula (I) as claimed in any one of claims 1, 2, 11 or 12, or pharmaceutically acceptable salts or solvates thereof, and a pharmaceutically acceptable diluent or carrier.

14. The pharmaceutical composition according to claim 13 further comprising at least one of the following active ingredients: prostaglandins; α-adrenergic receptor antagonist compounds; NO-agonist compounds; potassium channel openers or modulators; dopaminergic agents; vasodilator agents; thromboxane A2 agonists; CNS active agonists; orgot alkoloids; modulators of natruretic factors; angiotensin-converting enzyme inhibitors; angiotensin receptor antagonists; substrates for NO-synthase; calcium channel blockers; endothelin receptor antagonists; endothelin-converting enzyme inhibitors; cholesterol-lowering agents; antiplatelet agents; antithrombotic agents; insulin sensitizing agents; L-DOPA; carbidopa; acetylcholinesterase inhibitors; steroidal anti-inflammatory agents; non-steroidal anti-inflammatory agents; estrogen receptor modulators; estrogen agonists and/or antagonists; cannabinoid receptor modulators; NPY inhibitors; vasoactive intestinal protein; melanocortin receptor agonist or modulator or enhancer; serotonin receptor agonists, antagonist or modulator; androgen; oestrogen; modulator of transporters for noradrenaline, dopamine and or serotonin; purinergic receptor agonist and/or modulator; neurokinin receptor antagonists; opioid receptor agonist, antagonist or modulator; oxytocin/vasopressin receptor agonists or modulator or PDE inhibitors.

15. A method of treating or preventing a disorder or condition by inhibiting NEP in a mammal, comprising administering to said mammal a therapeutically effective amount of a compound of formula (I) as claimed in any one of claims 1, 2, 11 or 12, or a pharmaceutically acceptable salt, or solvate thereof.

16. The method according to claim 15 wherein R¹ is hydrogen.

17. The method according to claim 16 wherein R² is methyl.

18. The method according to claim 16 wherein R² is ethyl.

19. The method according to claim 15 wherein R¹ is methyl.

20. The method according to claim 19 wherein R² is methyl.

21. The method according to claim 20 wherein R² is ethyl.

22. The method according to claim 15 wherein R² is methyl.

23. The method according to claim 15 wherein R² is ethyl.

24. The method according to claim 15, wherein the disorder or condition is selected from hypertension, essential hypertension, pulmonary hypertension, secondary hypertension, isolated systolic hypertension, hypertension associated with diabetes, hypertension associated with atherosclerosis, and renovascular hypertension, peripheral vascular disease, heart failure, angina, renal insufficiency, acute renal failure, cyclical oedema, Menières disease, hyperaldosteroneism (primary and secondary), hypercalciuria, stroke, glaucoma, obesity, metabolic diseases, Metabolic Syndrome, diabetes, impaired glucose tolerance, diabetic retinopathy, diabetic neuropathy, menstrual disorders, preterm labour, pre-eclampsia, endometriosis, and reproductive disorders, male and female infertility, polycystic ovarian syndrome, implantation failure, asthma, inflammation, leukemia, pain, cancer pain, depression, drug abuse, cirrhosis, epilepsy, affective disorders, dementia and geriatric confusion, gastrointestinal disorders, diarrhoea, irritable bowel syndrome, wound healing, diabetic and venous ulcers and pressure sores, septic shock, gastric acid secretion, hyperreninaemia, cystic fibrosis, restenosis, athereosclerosis, female sexual dysfunction (FSD), sexual arousal disorder, female sexual arousal disorder (FSAD), male sexual dysfunction (MSD), male erectile dysfunction (MED), hypoactive sexual desire disorder, orgasmic disorder and sexual pain disorder.

25. The method according to claim 24 wherein the disorder or condition is selected from female sexual dysfunction (FSD), sexual arousal disorder, female sexual arousal disorder (FSAD), male sexual dysfunction (MSD), male erectile dysfunction (MED), hypoactive sexual desire disorder, orgasmic disorder and sexual pain disorder.

26. The method according to claim 25 wherein the disorder or condition is selected from female sexual dysfunction (FSD), female sexual arousal disorder (FSAD), male sexual dysfunction (MSD), and male erectile dysfunction (MED).

27. A method of treating or preventing a disorder or condition by inhibiting NEP in a mammal, comprising administering to said mammal a therapeutically effective amount of the composition as claimed in claim 14 or a pharmaceutically acceptable salt, or solvate thereof.

28. The method according to claim 27 wherein the disorder or condition is selected from female sexual dysfunction (FSD), female sexual arousal disorder (FSAD), male sexual dysfunction (MSD), and male erectile dysfunction (MED).

29. The method according to claim 28, wherein the disorder is FSD and the active ingredient is a
(a) PDE5 inhibitor selected from:
5-[2-ethoxy-5-(4-methyl-1-piperazinylsulphonyl)phenyl]-1-methyl-3-n-propyl-1,6-dihydro-7H-pyrazolo[4,3-d]pyrimidin-7-one sildenafil;
(6R,12aR)-2,3,6,7,12,12a-hexahydro-2-methyl-6-(3,4-methylene-dioxyphenyl)-pyrazino[2′,1′:6,1]pyrido[3,4-b]indole-1,4-dione;
2-[2-ethoxy-5-(4-ethyl-piperazin-1-yl-1-sulphonyl)-phenyl]-5-methyl-7-propyl-3H-imidazo[5,1-f][1,2,4]triazin-4-one;
5-[2-ethoxy-5-(4-ethylpiperazin-1-ylsulphonyl)pyridin-3-yl]-3-ethyl-2-[2-methoxyethyl]-2,6-dihydro-7H-pyrazolo[4,3-d]pyrimidin-7-one; and
5-(5-acetyl-2-butoxy-3-pyridinyl)-3-ethyl-2-(1-ethyl-3-azetidinyl)-2,6-dihydro-7H-pyrazolo[4,3-d]pyrimidin-7-one and pharmaceutically acceptable salts thereof;
(b) an NPY Y1 inhibitor;
(c) a dopamine agonist selected from apomorphine or a selective D₂, D₃ or D₂/D₃agonist selected from pramipexole and ropirinol;
(d) a melanocortin receptor agonist or modulator or melanocortin enhancer selected from melanotan 11, PT-14, PT-141;
(e) an agonist, antagonist or modulator for 5HT2C;
(f) an estrogen receptor modulator, agonist and/or antagonists selected from raloxifene, tibolone or lasofoxifene;
(g) an androgen selected from androsterone, dehydro-androsterone, testosterone, androstanedione or a synthetic androgen; and
(h) an oestrogen selected from oestradiol, oestrone, oestriol or synthetic estrogen.

30. The method according to claim 27 wherein the disorder is MED and the active ingredient is:
(a) a PDE5 inhibitor selected from:
5-[2-ethoxy-5-(4-methyl-1-piperazinylsulphonyl)phenyl]-1-methyl-3-n-propyl-1,6-dihydro-7H-pyrazolo[4,3-d]pyrimidin-7-one sildenafil;
(6R,12aR)-2,3,6,7,12,12a-hexahydro-2-methyl-6-(3,4-methylene-dioxyphenyl)-pyrazino[2′,1′:6,1]pyrido[3,4-b]indole-1,4-dione;
2-[2-ethoxy-5-(4-ethyl-piperazin-1-yl-1-sulphonyl)-phenyl]-5-methyl-7-propyl-3H-imidazo[5,1-f][1,2,4]triazin-4-one;
5-[2-ethoxy-5-(4-ethylpiperazin-1-ylsulphonyl)pyridin-3-yl]-3-ethyl-2-[2-methoxyethyl]-2,6-dihydro-7H-pyrazolo[4,3-d]pyrimidin-7-one; and
5-(5-acetyl-2-butoxy-3-pyridinyl)-3-ethyl-2-(1-ethyl-3-azetidinyl)-2,6-dihydro-7H-pyrazolo[4,3-d]pyrimidin-7-one and pharmaceutically acceptable salts thereof;
(b) an NPY Y1 inhibitor;
(c) a dopamine agonist selected from apomorphine or a selective D₂, D₃ or D₂/D₃agonist selected from pramipexole and ropirinol;
(d) a melanocortin receptor agonist or modulator or melanocortin enhancer selected from melanotan 11, PT-14, PT-141; or
(e) an agonist, antagonist or modulator for 5HT2C.

31. The method according to claim 27 wherein the condition is a cardiovascular disorder and the active ingredient is:
(a) angiotensin receptor blockers selected from losartan, valsartan, telmisartan, candesartan, irbesartan, eprosartan or olmesartan;
(b) calcium channel blockers selected from amlodipine;
(c) statins selected from atorvastatin;
(d) PDE5 inhbitors selected from sildenafil, tadalafil, vardenafil, 5-[2-ethoxy-5-(4-ethylpiperazin-1-ylsulphonyl)pyridin-3-yl]-3-ethyl-2-[2-methoxyethyl]-2,6-dihydro-7H-pyrazolo[4,3-d]pyrimidin-7-one; 5-(5-acetyl-2-butoxy-3-pyridinyl)-3-ethyl-2-(1-ethyl-3-azetidinyl)-2,6-dihydro-7H-pyrazolo[4,3-d]pyrimidin-7-one; or N-[[3-(4,7-dihydro-1-methyl-7-oxo-3-propyl-1H-pyrazolo[4,3-d]-pyrimidin-5-yl)-4-propxyphenyl]sulfonyl]-1-methyl2-pyrrolidinepropanamide;
(e) beta blockers selected from atenolol or carvedilol
(f) ACE inhibitors selected from quinapril, enalapril or lisinopril;
(g) alpha-blockers selected from doxazosin;
(h) selective aldosterone receptor antagonists selected from eplerenone or spironolactone; or
(i) imidazoline I₁ agonists selected from rilmenidine or moxonidine.

32. The method according to claims 27 wherein the composition is administered orally, bucally or sublingually and the therapeutically effective amount of the composition is 5 mg to 1000 mg.

33. The method according to claim 32 wherein the composition is administered orally.
